(19) United States
(12) Patent Application Publication
(10) Pub. No.: US 2023/0414673 A1
(43) Pub. Date: Dec. 28, 2023

(54) EXTRACELLULAR VESICLES DERIVED FROM CARDIOSPHERE-DERIVED CELLS AS ANTI-SHOCK THERAPEUTICS

(71) Applicants: Tiffani C. Chance, Fort Detrick, MD (US); CAPRICOR, INC, San Diego, CA (US); THE GOVERNMENT OF THE UNITED STATES AS REPRESENTED BY THE SECRETARY OF THE ARMY, Fort Detrick, MD (US)

(72) Inventors: Tiffani C. Chance, Castroville, TX (US); James A. Bynum, La Vernia, TX (US); Linda S. Marban, Santa Monica, CA (US)

(21) Appl. No.: 18/037,478

(22) PCT Filed: Nov. 23, 2021
(86) PCT No.: PCT/US21/60475

§ 371 Date: May 17, 2023

Related U.S. Application Data
(60) Provisional application No. 63/117,360, filed on Nov. 23, 2020.

(51) Int. Cl. A61K 35/34; A61K 9/127; A61P 7/04

(52) U.S. Cl. CPC A61K 35/34; A61K 9/127; A61P 7/04

(57) ABSTRACT

The present invention generally relates to the use of extracellular vesicles derived from cardiosphere-derived cells (CDC-EVs) as an anti-shock therapeutic. For instance, the present invention relates to a method of using CDC-EVs to treat polytrauma associated with coagulopathy and hemorrhagic shock in a subject in need thereof, wherein the lactate, glucose and/or creatinine levels in the subject are decreased upon being treated with a therapeutically effective amount of CDC-EVs. The results presented herein are of great relevance to the development of EV products for use in combat casualty care, as the studies presented herein show that CDC-EVs have the potential to be an anti-shock therapeutic if administered immediately after injury involving trauma associated with hemorrhagic shock and/or coagulopathy.

### REFERENCE TO RELATED APPLICATIONS

This application claims the benefit of the priority date of U.S. provisional application 63/117,360, filed Nov. 23, 2020, the contents of which are incorporated herein by reference in its entirety.

### BACKGROUND OF THE INVENTION

Polytrauma coupled with hemorrhagic shock is the worldwide leading cause of morbidity and mortality in those younger than 45 years of age.(1-3) Polytrauma and hemorrhagic shock typically affect two or more body segments, and thus present challenges for immediate transportation to the intensive care unit, diagnosis, management, and treatment.(2, 4-6) While patient outcome ultimately relies on immediate assessment and control of bleeding, the normalization of certain parameters (e.g. lactate and glucose levels; leukocyte levels) in the early hours of trauma can predict survival in trauma patients.(7-11) Serum lactate level, in particular, is a reliable, independent predictor of patient mortality, with lactate clearance within the first 24 hours correlating with reduced morbidity and mortality.(3, 9-12) Additionally, early lactate clearance has been shown to correlate with decreased levels of shock severity, while the persistence of elevated lactate levels leading to lactic acidosis can further depress organ function.(3, 13-15)

The lack of adequate supply of blood products, particularly in military environments, complicates the care of traumatically injured patients. Studies have shown that there has been a decline in blood collection and transfusion in the United States, as well as limited blood product supplies in low- and middle-income countries.(16, 17) Thus, there is a need for novel therapeutics in trauma care.

Studies have recently suggested that extracellular vesicles (EVs), comprised of microvesicles (200 nm to 1 μm in diameter; bud directly from the plasma membrane) and exosomes (30 to 200 nm; released internal vesicles of multivesicular bodies), could be useful in the treatment of multiple diseases.(18-23) This may be due to the retention in, and on, EVs of DNA, RNA, lipids, and proteins from their parent cells.(24-27) Alternative nomenclature is also often used to refer to EVs. Thus, as used herein the term “EVs” shall be given its ordinary meaning and may also include terms including exosomes, microvesicles, epididimosomes, argosomes, exosome-like vesicles, microparticles, promininosomes, prostasomes, dexosomes, texosomes, dex, tex, archeosomes and oncosomes. Exosomes are secreted by a wide range of mammalian cells and are secreted under both normal and pathological conditions. Exosomes function as intracellular messengers by virtue of carrying mRNA, miRNA or other contents from a first cell to another cell (or plurality of cells). In several embodiments, exosomes are involved in blood coagulation, immune modulation, metabolic regulation, cell division, and other cellular processes. Because of the wide variety of cells that secret exosomes, in several embodiments, exosome preparations can be used as a diagnostic tool (e.g., exosomes can be isolated from a particular tissue, evaluated for their nucleic acid or protein content, which can then be correlated to disease state or risk of developing a disease).

EVs derived from cardiosphere (self-assembling multicellular cluster from the cellular outgrowth from cardiac explants cultured in nonadhesive substrates)-derived cells (CDC-EVs) have been identified as a unique, cell-free therapeutic, with research focusing on their cardio protective and angiogenic potential.(28-31) While initial work on CDC-EVs is promising, there still exist significant gaps in knowledge regarding their therapeutic potential. Firstly, no work has been performed detailing their coagulation profile, which is an important parameter to consider before injection into patients with coagulopathy. Secondly, while CDC-EVs are beneficial in heart injury models, no work has been performed detailing their effects in more severe forms of trauma. More specifically, understanding the effects of CDC-EVs on the aforementioned parameters will be critical before more widespread use can be considered in both civilian and military populations.

The present inventors sought to evaluate the effects of CDC-EVs on coagulation system function to ensure safety in trauma patients who are coagulopathic and present risks of thrombosis. Prior in vitro work utilizing EVs isolated from mesenchymal stem cells (MSC-EVs) demonstrated that MSC-EVs display procoagulant activity associated with the surface expression of phosphatidylserine and tissue factor, and that further work is necessary to avoid adverse hemostatic effects related to MSC-EV therapy.(33, 34) Thus, failure to consider EV hemostatic profile could further exacerbate coagulopathy in trauma.(35, 36) Herein the present inventors describe for the first time the therapeutic potential of CDC-EVs in a rat model of polytrauma and hemorrhage by analyzing their in vitro hemostatic potential for therapeutic safety, then evaluating CDC-EVs in vivo by measuring effects on prothrombin times, lactate levels, and other indicators of trauma severity. Due to the severe nature of polytrauma and hemorrhagic shock, the present inventors were able to ignore any secondary regenerative benefits of CDC-EVs in favor of screening solely for an anti-shock therapeutic.

### SUMMARY OF THE INVENTION

The present invention provides, in a first aspect, a method of treating polytrauma in a mammalian subject in need thereof, the method comprising the step of administering to the subject a therapeutically effective amount of extracellular vesicles (EVs), e.g., EVs derived from cardiosphere-derived cells (CDC-EVs), immediately or within a short time period after polytrauma, wherein said polytrauma is associated with, or is coupled with, or is induced by, coagulopathy and hemorrhagic shock. In some embodiments, parameters selected from lactate, glucose and creatinine, and combinations thereof, that are monitored in the subject, are observed to decrease after treating the subject with the therapeutically effective amount of CDC-EVs. In some embodiments, the lactate level in the subject is decreased upon being treated with a therapeutically effective amount of extracellular vesicles, e.g., CDC-EVs. In some embodiments, the lactate and glucose levels in the subject are decreased upon being treated with a therapeutically effective amount of extracellular vesicles, e.g., CDC-EVs. In some embodiments, the lactate and creatinine levels in the subject are decreased upon being treated with a therapeutically effective amount of extracellular vesicles, e.g., CDC-EVs. In some embodiments, the lactate, glucose and creatinine levels in the subject are decreased upon being treated with a therapeutically effective amount of extracellular vesicles, e.g., CDC-EVs. In some embodiments, said effective amount of CDC-EVs is administered to the subject within 1, 2, 3, 4, 5, 6, 7, 8, 9, 10, 11, 12, 13, 14, 15, 16, 17, 18, 19, 20, 21, 22, 23, or 24 hours after polytrauma. In some embodiments, said therapeutically effective amount of CDC-EVs is administered to the subject systemically or locally. In some embodiments, said extracellular vesicles are exosomes, e.g., exosomes derived from cardiosphere-derived cells (CDC-derived exosomes). In some embodiments, are exosomes, microvesicles, membrane particles, membrane vesicles, exosome-like vesicles, ectosomes, ectosome-like vesicles, or exovesicles. In some embodiments, said mammalian subject is a human subject.

The present invention provides, in a second aspect, a method of decreasing the risks associated with polytrauma in a mammalian subject suffering from polytrauma comprising the step of: administering to the subject a therapeutically effective amount of extracellular vesicles (EVs), e.g., EVs derived from cardiosphere-derived cells (CDC-EVs), immediately or within a short time period after polytrauma, wherein said polytrauma is associated with coagulopathy and/or hemorrhagic shock. In some embodiments, the lactate level in the subject is decreased upon being treated with a therapeutically effective amount of extracellular vesicles, e.g., CDC-EVs. In some embodiments, the lactate and glucose levels in the subject are decreased upon being treated with a therapeutically effective amount of extracellular vesicles, e.g., CDC-EVs. In some embodiments, the lactate and creatinine levels in the subject are decreased upon being treated with a therapeutically effective amount of extracellular vesicles, e.g., CDC-EVs. In some embodiments, the lactate, glucose and creatinine levels in the subject are decreased upon being treated with a therapeutically effective amount of extracellular vesicles, e.g., CDC-EVs. In some embodiments, said effective amount of extracellular vesicles, e.g., CDC-EVs, is administered to the subject immediately after polytrauma, hemorrhagic shock and/or coagulopathy. In some embodiments, said effective amount of CDC-EVs is administered to the subject within 1, 2, 3, 4, 5, 6, 7, 8, 9, 10, 11, 12, 13, 14, 15, 16, 17, 18, 19, 20, 21, 22, 23, or 24 hours after polytrauma, hemorrhagic shock and/or coagulopathy. In some embodiments, said therapeutically effective amount of CDC-EVs is administered to the subject systemically or locally. In some embodiments, said extracellular vesicles are exosomes, e.g., exosomes derived from cardiosphere-derived cells (CDC-derived exosomes). In some embodiments, are exosomes, microvesicles, membrane particles, membrane vesicles, exosome-like vesicles, ectosomes, ectosome-like vesicles, or exovesicles. In some embodiments, said subject is a mammalian subject, e.g., a human subject.

The present invention provides, in a third aspect, a method of treating trauma in a subject in need thereof, the method comprising the step of administering to the subject a therapeutically effective amount of extracellular vesicles, e.g., extracellular vesicles derived from cardiosphere-derived cells (CDC-EVs). In some embodiments, said trauma is associated with, or is coupled with, or is induced by, hemorrhagic shock. In some embodiments, said trauma is associated with, or is coupled with, or is induced by, coagulopathy. In some embodiments, said trauma is associated with, or is coupled with, or is induced by, coagulopathy and hemorrhagic shock. In some embodiments, said trauma is polytrauma. In some embodiments, said trauma is acute trauma, e.g., acute polytrauma. In some embodiments, the lactate level in the subject is decreased upon being treated with a therapeutically effective amount of extracellular vesicles, e.g., CDC-EVs. In some embodiments, the lactate and glucose levels in the subject are decreased upon being treated with a therapeutically effective amount of extracellular vesicles, e.g., CDC-EVs. In some embodiments, the lactate and creatinine levels in the subject are decreased upon being treated with a therapeutically effective amount of extracellular vesicles, e.g., CDC-EVs. In some embodiments, the lactate, glucose and creatinine levels in the subject are decreased upon being treated with a therapeutically effective amount of extracellular vesicles, e.g., CDC-EVs. In some embodiments, said effective amount of extracellular vesicles, e.g., CDC-EVs, is administered to the subject immediately or within a short time period after trauma, hemorrhagic shock and/or coagulopathy. In some embodiments, said effective amount of CDC-EVs is administered to the subject within 1, 2, 3, 4, 5, 6, 7, 8, 9, 10, 11, 12, 13, 14, 15, 16, 17, 18, 19, 20, 21, 22, 23, or 24 hours after trauma, hemorrhagic shock and/or coagulopathy. In some embodiments, said therapeutically effective amount of CDC-EVs is administered to the subject systemically or locally. In some embodiments, said extracellular vesicles are exosomes, e.g., exosomes derived from cardiosphere-derived cells (CDC-derived exosomes). In some embodiments, are exosomes, microvesicles, membrane particles, membrane vesicles, exosome-like vesicles, ectosomes, ectosome-like vesicles, or exovesicles. In some embodiments, said subject is a mammalian subject, e.g., a human subject. In some embodiments, said trauma comprises one or more organ system failure, e.g., liver and/or kidney failure.

The present invention provides, in a fourth aspect, a method of treating coagulopathy in a subject in need thereof, the method comprising the step of administering to the subject a therapeutically effective amount of extracellular vesicles, e.g., extracellular vesicles derived from cardiosphere-derived cells (CDC-EVs). In some embodiments, said coagulopathy is acute coagulopathy. In some embodiments, said coagulopathy is associated with, or is coupled with, or is induced by, hemorrhagic shock. In some embodiments, said coagulopathy is associated with, or is coupled with, or is induced by, trauma. In some embodiments, said coagulopathy is associated with, or is coupled with, or is induced by, trauma and hemorrhagic shock. In some embodiments, said trauma is polytrauma. In some embodiments, said trauma is acute trauma, e.g., acute polytrauma. In some embodiments, the lactate level in the subject is decreased upon being treated with a therapeutically effective amount of extracellular vesicles, e.g., CDC-EVs. In some embodiments, the lactate and glucose levels in the subject are decreased upon being treated with a therapeutically effective amount of extracellular vesicles, e.g., CDC-EVs. In some embodiments, the lactate and creatinine levels in the subject are decreased upon being treated with a therapeutically effective amount of extracellular vesicles, e.g., CDC-EVs. In some embodiments, the lactate, glucose and creatinine levels in the subject are decreased upon being treated with a therapeutically effective amount of extracellular vesicles, e.g., CDC-EVs. In some embodiments, said effective amount of extracellular vesicles, e.g., CDC-EVs, is administered to the subject immediately or within a short time period after coagulopathy, hemorrhagic shock and/or trauma. In some embodiments, said effective amount of CDC-EVs is administered to the subject within 1, 2, 3, 4, 5, 6, 7, 8, 9, 10, 11, 12, 13, 14, 15, 16, 17, 18, 19, 20, 21, 22, 23, or 24 hours after coagulopathy, hemorrhagic shock and/or trauma. In some embodiments, said therapeutically effective amount of CDC-EVs is administered to the subject systemically or locally. In some embodiments, said extracellular vesicles are exosomes, e.g., exosomes derived from cardiosphere-derived cells (CDC-derived exosomes). In some embodiments, are exosomes, microvesicles, membrane particles, membrane vesicles, exosome-like vesicles, ectosomes, ectosome-like vesicles, or exovesicles. In some embodiments, said subject is a mammalian subject, e.g., a human subject. In some embodiments, said trauma comprises one or more organ system failure, e.g., liver and/or kidney failure.

The present invention provides, in a fifth aspect, a method of decreasing lactate, glucose and/or creatinine levels in a subject suffering from trauma, the method comprising administering to the subject a therapeutically effective amount of extracellular vesicles, e.g., extracellular vesicles derived from cardiosphere-derived cells (CDC-EVs). In some embodiments, said trauma is associated with, or is coupled with, or is induced by, hemorrhagic shock. In some embodiments, said trauma is associated with, or is coupled with, or is induced by, coagulopathy. In some embodiments, said trauma is associated with, or is coupled with, or is induced by, coagulopathy and hemorrhagic shock. In some embodiments, said trauma is polytrauma. In some embodiments, said trauma is acute trauma, e.g., acute polytrauma. In some embodiments, the lactate level in the subject is decreased upon being treated with a therapeutically effective amount of extracellular vesicles, e.g., CDC-EVs. In some embodiments, the lactate and glucose levels in the subject are decreased upon being treated with a therapeutically effective amount of extracellular vesicles, e.g., CDC-EVs. In some embodiments, the lactate and creatinine levels in the subject are decreased upon being treated with a therapeutically effective amount of extracellular vesicles, e.g., CDC-EVs. In some embodiments, the lactate, glucose and creatinine levels in the subject are decreased upon being treated with a therapeutically effective amount of extracellular vesicles, e.g., CDC-EVs. In some embodiments, said effective amount of extracellular vesicles, e.g., CDC-EVs, is administered to the subject immediately or within a short time period after trauma, hemorrhagic shock and/or coagulopathy. In some embodiments, said effective amount of extracellular vesicles, e.g., CDC-EVs, is administered to the subject within 1, 2, 3, 4, 5, 6, 7, 8, 9, 10, 11, 12, 13, 14, 15, 16, 17, 18, 19, 20, 21, 22, 23, or 24 hours after trauma, hemorrhagic shock and/or coagulopathy. In some embodiments, said therapeutically effective amount of CDC-EVs is administered to the subject systemically or locally. In some embodiments, said extracellular vesicles are exosomes, e.g., exosomes derived from cardiosphere-derived cells (CDC-derived exosomes). In some embodiments, are exosomes, microvesicles, membrane particles, membrane vesicles, exosome-like vesicles, ectosomes, ectosome-like vesicles, or exovesicles. In some embodiments, said subject is a mammalian subject, e.g., a human subject. In some embodiments, said trauma comprises one or more organ system failure, e.g., liver and/or kidney failure.

The present invention provides, in a sixth aspect, a method of decreasing lactate, glucose and/or creatinine levels in a subject suffering from coagulopathy, the method comprising administering to the subject a therapeutically effective amount of extracellular vesicles, e.g., extracellular vesicles derived from cardiosphere-derived cells (CDC-EVs). In some embodiments, said coagulopathy is acute coagulopathy. In some embodiments, said coagulopathy is associated with, or is coupled with, or is induced by, hemorrhagic shock. In some embodiments, said coagulopathy is associated with, or is coupled with, or is induced by, trauma. In some embodiments, said coagulopathy is associated with, or is coupled with, or is induced by, trauma and hemorrhagic shock. In some embodiments, said trauma is associated with, or is coupled with, or is induced by, coagulopathy and hemorrhagic shock. In some embodiments, said trauma is polytrauma. In some embodiments, said trauma is acute trauma, e.g., acute polytrauma. In some embodiments, the lactate level in the subject is decreased upon being treated with a therapeutically effective amount of extracellular vesicles, e.g., CDC-EVs. In some embodiments, the lactate and glucose levels in the subject are decreased upon being treated with a therapeutically effective amount of extracellular vesicles, e.g., CDC-EVs. In some embodiments, the lactate and creatinine levels in the subject are decreased upon being treated with a therapeutically effective amount of extracellular vesicles, e.g., CDC-EVs. In some embodiments, the lactate, glucose and creatinine levels in the subject are decreased upon being treated with a therapeutically effective amount of extracellular vesicles, e.g., CDC-EVs. In some embodiments, said effective amount of extracellular vesicles, e.g., CDC-EVs, is administered to the subject immediately or within a short time period after coagulopathy, hemorrhagic shock and/or trauma. In some embodiments, said effective amount of CDC-EVs is administered to the subject within 1, 2, 3, 4, 5, 6, 7, 8, 9, 10, 11, 12, 13, 14, 15, 16, 17, 18, 19, 20, 21, 22, 23, or 24 hours after coagulopathy, hemorrhagic shock and/or trauma. In some embodiments, said therapeutically effective amount of CDC-EVs is administered to the subject systemically or locally. In some embodiments, said extracellular vesicles are exosomes, e.g., exosomes derived from cardiosphere-derived cells (CDC-derived exosomes). In some embodiments, are exosomes, microvesicles, membrane particles, membrane vesicles, exosome-like vesicles, ectosomes, ectosome-like vesicles, or exovesicles. In some embodiments, said subject is a mammalian subject, e.g., a human subject. In some embodiments, said trauma comprises one or more organ system failure, e.g., liver and/or kidney failure.

The present invention provides, in a seventh aspect, a method of decreasing the risk of shock in a subject suffering from trauma, the method comprising administering to the subject a therapeutically effective amount of extracellular vesicles, e.g., extracellular vesicles derived from cardiosphere-derived cells (CDC-EVs). In some embodiments, said trauma is associated with, or is coupled with, or is induced by, hemorrhagic shock. In some embodiments, said trauma is associated with, or is coupled with, or is induced by, coagulopathy. In some embodiments, said trauma is associated with, or is coupled with, or is induced by, coagulopathy and hemorrhagic shock. In some embodiments, said trauma is polytrauma. In some embodiments, said trauma is acute trauma, e.g., acute polytrauma. In some embodiments, the lactate level in the subject is decreased upon being treated with a therapeutically effective amount of extracellular vesicles, e.g., CDC-EVs. In some embodiments, the lactate and glucose levels in the subject are decreased upon being treated with a therapeutically effective amount of extracellular vesicles, e.g., CDC-EVs. In some embodiments, the lactate and creatinine levels in the subject are decreased upon being treated with a therapeutically effective amount of extracellular vesicles, e.g., CDC-EVs. In some embodiments, the lactate, glucose and creatinine levels in the subject are decreased upon being treated with a therapeutically effective amount of extracellular vesicles, e.g., CDC-EVs. In some embodiments, said effective amount of extracellular vesicles, e.g., CDC-EVs, is administered to the subject immediately or within a short time period after trauma, hemorrhagic shock and/or coagulopathy. In some embodiments, said effective amount of CDC-EVs is administered to the subject within 1, 2, 3, 4, 5, 6, 7, 8, 9, 10, 11, 12, 13, 14, 15, 16, 17, 18, 19, 20, 21, 22, 23, or 24 hours after trauma, hemorrhagic shock and/or coagulopathy. In some embodiments, said therapeutically effective amount of CDC-EVs is administered to the subject systemically or locally. In some embodiments, said extracellular vesicles are exosomes, e.g., exosomes derived from cardiosphere-derived cells (CDC-derived exosomes). In some embodiments, are exosomes, microvesicles, membrane particles, membrane vesicles, exosome-like vesicles, ectosomes, ectosome-like vesicles, or exovesicles. In some embodiments, said subject is a mammalian subject, e.g., a human subject. In some embodiments, said trauma comprises one or more organ system failure, e.g., liver and/or kidney failure.

The present invention provides, in an eighth aspect, a method of decreasing the risk of shock in a subject suffering from coagulopathy, the method comprising administering to the subject a therapeutically effective amount of extracellular vesicles, e.g., extracellular vesicles derived from cardiosphere-derived cells (CDC-EVs). In some embodiments, said coagulopathy is acute coagulopathy. In some embodiments, said coagulopathy is associated with, or is coupled with, or is induced by, hemorrhagic shock. In some embodiments, said coagulopathy is associated with, or is coupled with, or is induced by, trauma. In some embodiments, said coagulopathy is associated with, or is coupled with, or is induced by, trauma and hemorrhagic shock. In some embodiments, said trauma is polytrauma. In some embodiments, said trauma is acute trauma, e.g., acute polytrauma. In some embodiments, the lactate level in the subject is decreased upon being treated with a therapeutically effective amount of extracellular vesicles, e.g., CDC-EVs. In some embodiments, the lactate and glucose levels in the subject are decreased upon being treated with a therapeutically effective amount of extracellular vesicles, e.g., CDC-EVs. In some embodiments, the lactate and creatinine levels in the subject are decreased upon being treated with a therapeutically effective amount of extracellular vesicles, e.g., CDC-EVs. In some embodiments, the lactate, glucose and creatinine levels in the subject are decreased upon being treated with a therapeutically effective amount of extracellular vesicles, e.g., CDC-EVs. In some embodiments, said effective amount of extracellular vesicles, e.g., CDC-EVs, is administered to the subject immediately or within a short time period after coagulopathy, hemorrhagic shock and/or trauma. In some embodiments, said effective amount of extracellular vesicles, e.g., CDC-EVs, is administered to the subject within 1, 2, 3, 4, 5, 6, 7, 8, 9, 10, 11, 12, 13, 14, 15, 16, 17, 18, 19, 20, 21, 22, 23, or 24 hours after coagulopathy, hemorrhagic shock and/or trauma. In some embodiments, said therapeutically effective amount of CDC-EVs is administered to the subject systemically or locally. In some embodiments, said extracellular vesicles are exosomes, e.g., exosomes derived from cardiosphere-derived cells (CDC-derived exosomes). In some embodiments, are exosomes, microvesicles, membrane particles, membrane vesicles, exosome-like vesicles, ectosomes, ectosome-like vesicles, or exovesicles. In some embodiments, said subject is a mammalian subject, e.g., a human subject. In some embodiments, said trauma comprises one or more organ system failure, e.g., liver and/or kidney failure.

The present invention provides, in a ninth aspect, a method of treating trauma to an organ in a subject in need thereof, the method comprising administering to the subject a therapeutically effective amount of extracellular vesicles, e.g., extracellular vesicles derived from cardiosphere-derived cells (CDC-EVs). In some embodiments, said organ is selected from the liver, kidneys, intestines, heart, arteries, veins, stomach, lungs, brain, spinal cord, cerebellum, nerves, placenta, uterus, spleen, bladder, esophagus, pancreas, colon, rectum, sex organs, pharynx, larynx, trachea, bronchi, salivary glands, diaphragm, skeletal muscles, bones, bone marrow, cartilage, ligaments, tendons, lymphatic vessels, ureters, urethra, endocrine glands, blood vessels, and gallbladder. In some embodiments, said organ is the liver. In some embodiments, said organ is one or both of the kidneys. In some embodiments, said organ is the liver and one or both of the kidneys. In some embodiments, said trauma is associated with, or is coupled with, or is induced by, hemorrhagic shock. In some embodiments, said trauma is associated with, or is coupled with, or is induced by, coagulopathy. In some embodiments, said trauma is associated with, or is coupled with, or is induced by, coagulopathy and hemorrhagic shock. In some embodiments, said trauma is polytrauma. In some embodiments, said trauma is acute trauma, e.g., acute polytrauma. In some embodiments, the lactate level in the subject is decreased upon being treated with a therapeutically effective amount of extracellular vesicles, e.g., CDC-EVs. In some embodiments, the lactate and glucose levels in the subject are decreased upon being treated with a therapeutically effective amount of extracellular vesicles, e.g., CDC-EVs. In some embodiments, the lactate and creatinine levels in the subject are decreased upon being treated with a therapeutically effective amount of extracellular vesicles, e.g., CDC-EVs. In some embodiments, the lactate, glucose and creatinine levels in the subject are decreased upon being treated with a therapeutically effective amount of extracellular vesicles, e.g., CDC-EVs. In some embodiments, said effective amount of extracellular vesicles, e.g., CDC-EVs, is administered to the subject immediately or within a short time period after trauma, coagulopathy and/or hemorrhagic shock. In some embodiments, said effective amount of extracellular vesicles, e.g., CDC-EVs, is administered to the subject within 1, 2, 3, 4, 5, 6, 7, 8, 9, 10, 11, 12, 13, 14, 15, 16, 17, 18, 19, 20, 21, 22, 23, or 24 hours after trauma, coagulopathy and/or hemorrhagic shock. In some embodiments, said therapeutically effective amount of CDC-EVs is administered to the subject systemically or locally. In some embodiments, said extracellular vesicles are exosomes, e.g., exosomes derived from cardiosphere-derived cells (CDC-derived exosomes). In some embodiments, are exosomes, microvesicles, membrane particles, membrane vesicles, exosome-like vesicles, ectosomes, ectosome-like vesicles, or exovesicles. In some embodiments, said subject is a mammalian subject, e.g., a human subject.

The present invention provides, in a tenth aspect, a formulation comprising extracellular vesicles (EVs), e.g., EVs derived from cardiosphere-derived cells (CDC-EVs), and at least one pharmaceutically acceptable carrier for use in treating trauma in a subject in need thereof. In some embodiments, said trauma is associated with, or is coupled with, or is induced by, hemorrhagic shock. In some embodiments, said trauma is associated with, or is coupled with, or is induced by, coagulopathy. In some embodiments, said trauma is associated with, or is coupled with, or is induced by, hemorrhagic shock and coagulopathy. In some embodiments, said trauma is polytrauma. In some embodiments, said trauma is acute trauma, e.g., acute polytrauma. In some embodiments, the lactate level in the subject is decreased upon being treated with a therapeutically effective amount of extracellular vesicles, e.g., CDC-EVs. In some embodiments, the lactate and glucose levels in the subject are decreased upon being treated with a therapeutically effective amount of extracellular vesicles, e.g., CDC-EVs. In some embodiments, the lactate and creatinine levels in the subject are decreased upon being treated with a therapeutically effective amount of extracellular vesicles, e.g., CDC-EVs. In some embodiments, the lactate, glucose and creatinine levels in the subject are decreased upon being treated with a therapeutically effective amount of extracellular vesicles, e.g., CDC-EVs. In some embodiments, said effective amount of extracellular vesicles, e.g., CDC-EVs, is administered to the subject immediately or within a short time period after trauma, hemorrhagic shock and/or coagulopathy. In some embodiments, said effective amount of extracellular vesicles, e.g., CDC-EVs, is administered to the subject within 1, 2, 3, 4, 5, 6, 7, 8, 9, 10, 11, 12, 13, 14, 15, 16, 17, 18, 19, 20, 21, 22, 23, or 24 hours after trauma, hemorrhagic shock and/or coagulopathy. In some embodiments, said therapeutically effective amount of CDC-EVs is administered to the subject systemically or locally. In some embodiments, said extracellular vesicles are exosomes, e.g., exosomes derived from cardiosphere-derived cells (CDC-derived exosomes). In some embodiments, are exosomes, microvesicles, membrane particles, membrane vesicles, exosome-like vesicles, ectosomes, ectosome-like vesicles, or exovesicles. In some embodiments, said subject is a mammalian subject, e.g., a human subject. In some embodiments, said trauma comprises one or more organ system failure, e.g., liver and/or kidney failure. In some embodiments, said formulation is suitable for use in combat casualty care. In some embodiments, said formulation is suitable for use as an anti-shock therapeutic.

The present invention provides, in an eleventh aspect, a formulation comprising extracellular vesicles (EVs), e.g., EVs derived from cardiosphere-derived cells (CDC-EVs), for use in treating trauma in a subject in need thereof. In some embodiments, said trauma is associated with, or is coupled with, or is induced by, hemorrhagic shock. In some embodiments, said trauma is associated with, or is coupled with, or is induced by, coagulopathy. In some embodiments, said trauma is associated with, or is coupled with, or is induced by, hemorrhagic shock and coagulopathy. In some embodiments, said trauma is polytrauma. In some embodiments, said trauma is acute trauma, e.g., acute polytrauma. In some embodiments, the lactate level in the subject is decreased upon being treated with a therapeutically effective amount of extracellular vesicles, e.g., CDC-EVs. In some embodiments, the lactate and glucose levels in the subject are decreased upon being treated with a therapeutically effective amount of extracellular vesicles, e.g., CDC-EVs. In some embodiments, the lactate and creatinine levels in the subject are decreased upon being treated with a therapeutically effective amount of extracellular vesicles, e.g., CDC-EVs. In some embodiments, the lactate, glucose and creatinine levels in the subject are decreased upon being treated with a therapeutically effective amount of extracellular vesicles, e.g., CDC-EVs. In some embodiments, said effective amount of extracellular vesicles, e.g., CDC-EVs, is administered to the subject immediately or within a short time period after trauma, hemorrhagic shock and/or coagulopathy. In some embodiments, said effective amount of extracellular vesicles, e.g., CDC-EVs, is administered to the subject within 1, 2, 3, 4, 5, 6, 7, 8, 9, 10, 11, 12, 13, 14, 15, 16, 17, 18, 19, 20, 21, 22, 23, or 24 hours after trauma, hemorrhagic shock and/or coagulopathy. In some embodiments, said therapeutically effective amount of CDC-EVs is administered to the subject systemically or locally. In some embodiments, said extracellular vesicles are exosomes, e.g., exosomes derived from cardiosphere-derived cells (CDC-derived exosomes). In some embodiments, are exosomes, microvesicles, membrane particles, membrane vesicles, exosome-like vesicles, ectosomes, ectosome-like vesicles, or exovesicles. In some embodiments, said subject is a mammalian subject, e.g., a human subject. In some embodiments, said trauma comprises one or more organ system failure, e.g., liver and/or kidney failure. In some embodiments, said formulation is suitable for use in combat casualty care. In some embodiments, said formulation is suitable for use as an anti-shock therapeutic.

The present invention provides, in a twelfth aspect, a formulation comprising extracellular vesicles (EVs), e.g., EVs derived from cardiosphere-derived cells (CDC-EVs), for use in treating coagulopathy in a subject in need thereof. In some embodiments, said coagulopathy is acute coagulopathy. In some embodiments, said coagulopathy is associated with, or is coupled with, or is induced by, hemorrhagic shock. In some embodiments, said coagulopathy is associated with, or is coupled with, or is induced by, trauma. In some embodiments, said coagulopathy is associated with, or is coupled with, or is induced by, trauma and hemorrhagic shock. In some embodiments, said trauma is polytrauma. In some embodiments, said trauma is acute trauma, e.g., acute polytrauma. In some embodiments, the lactate level in the subject is decreased upon being treated with a therapeutically effective amount of extracellular vesicles, e.g., CDC-EVs. In some embodiments, the lactate and glucose levels in the subject are decreased upon being treated with a therapeutically effective amount of extracellular vesicles, e.g., CDC-EVs. In some embodiments, the lactate and creatinine levels in the subject are decreased upon being treated with a therapeutically effective amount of extracellular vesicles, e.g., CDC-EVs. In some embodiments, the lactate, glucose and creatinine levels in the subject are decreased upon being treated with a therapeutically effective amount of extracellular vesicles, e.g., CDC-EVs. In some embodiments, said effective amount of extracellular vesicles, e.g., CDC-EVs, is administered to the subject immediately or within a short time period after coagulopathy, hemorrhagic shock and/or trauma. In some embodiments, said effective amount of extracellular vesicles, e.g., CDC-EVs, is administered to the subject within 1, 2, 3, 4, 5, 6, 7, 8, 9, 10, 11, 12, 13, 14, 15, 16, 17, 18, 19, 20, 21, 22, 23, or 24 hours after coagulopathy, hemorrhagic shock and/or trauma. In some embodiments, said therapeutically effective amount of CDC-EVs is administered to the subject systemically or locally. In some embodiments, said extracellular vesicles are exosomes, e.g., exosomes derived from cardiosphere-derived cells (CDC-derived exosomes). In some embodiments, are exosomes, microvesicles, membrane particles, membrane vesicles, exosome-like vesicles, ectosomes, ectosome-like vesicles, or exovesicles. In some embodiments, said subject is a mammalian subject, e.g., a human subject. In some embodiments, said trauma comprises one or more organ system failure, e.g., liver and/or kidney failure. In some embodiments, said formulation is suitable for use in combat casualty care. In some embodiments, said formulation is suitable for use as an anti-shock therapeutic.

The present invention provides, in a thirteenth aspect, a formulation comprising extracellular vesicles (EVs), e.g., EVs derived from cardiosphere-derived cells (CDC-EVs), for use in a method of decreasing the lactate, glucose and/or creatinine levels in a subject suffering from trauma, the method comprising administering to the subject a therapeutically effective amount of CDC-EVs. In some embodiments, said trauma is associated with, or is coupled with, or is induced by, hemorrhagic shock. In some embodiments, said trauma is associated with, or is coupled with, or is induced by, coagulopathy. In some embodiments, said trauma is associated with, or is coupled with, or is induced by, coagulopathy and hemorrhagic shock. In some embodiments, said trauma is polytrauma. In some embodiments, said trauma is acute trauma, e.g., acute polytrauma. In some embodiments, the lactate level in the subject is decreased upon being treated with a therapeutically effective amount of extracellular vesicles, e.g., CDC-EVs. In some embodiments, the lactate and glucose levels in the subject are decreased upon being treated with a therapeutically effective amount of extracellular vesicles, e.g., CDC-EVs. In some embodiments, the lactate and creatinine levels in the subject are decreased upon being treated with a therapeutically effective amount of extracellular vesicles, e.g., CDC-EVs. In some embodiments, the lactate, glucose and creatinine levels in the subject are decreased upon being treated with a therapeutically effective amount of extracellular vesicles, e.g., CDC-EVs. In some embodiments, said effective amount of extracellular vesicles, e.g., CDC-EVs, is administered to the subject immediately or within a short time period after trauma, hemorrhagic shock and/or coagulopathy. In some embodiments, said effective amount of extracellular vesicles, e.g., CDC-EVs, is administered to the subject within 1, 2, 3, 4, 5, 6, 7, 8, 9, 10, 11, 12, 13, 14, 15, 16, 17, 18, 19, 20, 21, 22, 23, or 24 hours after trauma, hemorrhagic shock and/or coagulopathy. In some embodiments, said therapeutically effective amount of CDC-EVs is administered to the subject systemically or locally. In some embodiments, said extracellular vesicles are exosomes, e.g., exosomes derived from cardiosphere-derived cells (CDC-derived exosomes). In some embodiments, are exosomes, microvesicles, membrane particles, membrane vesicles, exosome-like vesicles, ectosomes, ectosome-like vesicles, or exovesicles. In some embodiments, said subject is a mammalian subject, e.g., a human subject. In some embodiments, said trauma comprises one or more organ system failure, e.g., liver and/or kidney failure. In some embodiments, said formulation is suitable for use in combat casualty care. In some embodiments, said formulation is suitable for use as an anti-shock therapeutic.

The present invention provides, in a fourteenth aspect, a formulation comprising extracellular vesicles (EVs), e.g., EVs derived from cardiosphere-derived cells (CDC-EVs), for use in a method of decreasing the lactate, glucose and/or creatinine levels in a subject suffering from coagulopathy, the method comprising administering to the subject a therapeutically effective amount of CDC-EVs. In some embodiments, said coagulopathy is acute coagulopathy. In some embodiments, said coagulopathy is associated with, or is coupled with, or is induced by, hemorrhagic shock. In some embodiments, said coagulopathy is associated with, or is coupled with, or is induced by, trauma. In some embodiments, said coagulopathy is associated with, or is coupled with, or is induced by, coagulopathy and hemorrhagic shock. In some embodiments, said trauma is polytrauma. In some embodiments, said trauma is acute trauma, e.g., acute polytrauma. In some embodiments, the lactate level in the subject is decreased upon being treated with a therapeutically effective amount of extracellular vesicles, e.g., CDC-EVs. In some embodiments, the lactate and glucose levels in the subject are decreased upon being treated with a therapeutically effective amount of extracellular vesicles, e.g., CDC-EVs. In some embodiments, the lactate and creatinine levels in the subject are decreased upon being treated with a therapeutically effective amount of extracellular vesicles, e.g., CDC-EVs. In some embodiments, the lactate, glucose and creatinine levels in the subject are decreased upon being treated with a therapeutically effective amount of extracellular vesicles, e.g., CDC-EVs. In some embodiments, said effective amount of extracellular vesicles, e.g., CDC-EVs, is administered to the subject immediately or within a short time period after coagulopathy, hemorrhagic shock and/or trauma. In some embodiments, said effective amount of extracellular vesicles, e.g., CDC-EVs, is administered to the subject within 1, 2, 3, 4, 5, 6, 7, 8, 9, 10, 11, 12, 13, 14, 15, 16, 17, 18, 19, 20, 21, 22, 23, or 24 hours after coagulopathy, hemorrhagic shock and/or trauma. In some embodiments, said therapeutically effective amount of CDC-EVs is administered to the subject systemically or locally. In some embodiments, said extracellular vesicles are exosomes, e.g., exosomes derived from cardiosphere-derived cells (CDC-derived exosomes). In some embodiments, are exosomes, microvesicles, membrane particles, membrane vesicles, exosome-like vesicles, ectosomes, ectosome-like vesicles, or exovesicles. In some embodiments, said subject is a mammalian subject, e.g., a human subject. In some embodiments, said trauma comprises one or more organ system failure, e.g., liver and/or kidney failure. In some embodiments, said formulation is suitable for use in combat casualty care. In some embodiments, said formulation is suitable for use as an anti-shock therapeutic.

The present invention provides, in a fifteenth aspect, a formulation comprising extracellular vesicles (EVs), e.g., EVs derived from cardiosphere-derived cells (CDC-EVs), for use in a method of decreasing the risk of shock in a subject suffering from trauma, the method comprising administering to the subject a therapeutically effective amount of CDC-EVs. In some embodiments, said trauma is associated with, or is coupled with, or is induced by, hemorrhagic shock. In some embodiments, said trauma is associated with, or is coupled with, or is induced by, coagulopathy. In some embodiments, said trauma is associated with, or is coupled with, or is induced by, hemorrhagic shock and coagulopathy. In some embodiments, said trauma is polytrauma. In some embodiments, said trauma is acute trauma, e.g., acute polytrauma. In some embodiments, the lactate level in the subject is decreased upon being treated with a therapeutically effective amount of extracellular vesicles, e.g., CDC-EVs. In some embodiments, the lactate and glucose levels in the subject are decreased upon being treated with a therapeutically effective amount of extracellular vesicles, e.g., CDC-EVs. In some embodiments, the lactate and creatinine levels in the subject are decreased upon being treated with a therapeutically effective amount of extracellular vesicles, e.g., CDC-EVs. In some embodiments, the lactate, glucose and creatinine levels in the subject are decreased upon being treated with a therapeutically effective amount of extracellular vesicles, e.g., CDC-EVs. In some embodiments, said effective amount of extracellular vesicles, e.g., CDC-EVs, is administered to the subject immediately or within a short time period after trauma, hemorrhagic shock and/or coagulopathy. In some embodiments, said effective amount of extracellular vesicles, e.g., CDC-EVs, is administered to the subject within 1, 2, 3, 4, 5, 6, 7, 8, 9, 10, 11, 12, 13, 14, 15, 16, 17, 18, 19, 20, 21, 22, 23, or 24 hours after trauma, hemorrhagic shock and/or coagulopathy. In some embodiments, said therapeutically effective amount of CDC-EVs is administered to the subject systemically or locally. In some embodiments, said extracellular vesicles are exosomes, e.g., exosomes derived from cardiosphere-derived cells (CDC-derived exosomes). In some embodiments, are exosomes, microvesicles, membrane particles, membrane vesicles, exosome-like vesicles, ectosomes, ectosome-like vesicles, or exovesicles. In some embodiments, said subject is a mammalian subject, e.g., a human subject. In some embodiments, said trauma comprises one or more organ system failure, e.g., liver and/or kidney failure. In some embodiments, said formulation is suitable for use in combat casualty care. In some embodiments, said formulation is suitable for use as an anti-shock therapeutic.

The present invention provides, in a sixteenth aspect, a formulation comprising extracellular vesicles (EVs), e.g., EVs derived from cardiosphere-derived cells (CDC-EVs), for use in a method of decreasing the risk of shock in a subject suffering from coagulopathy, the method comprising administering to the subject a therapeutically effective amount of CDC-EVs. In some embodiments, said coagulopathy is acute coagulopathy. In some embodiments, said coagulopathy is associated with, or is coupled with, or is induced by, hemorrhagic shock. In some embodiments, said coagulopathy is associated with, or is coupled with, or is induced by, trauma. In some embodiments, said coagulopathy is associated with, or is coupled with, or is induced by, trauma and hemorrhagic shock. In some embodiments, said trauma is polytrauma. In some embodiments, said trauma is acute trauma, e.g., acute polytrauma. In some embodiments, the lactate level in the subject is decreased upon being treated with a therapeutically effective amount of extracellular vesicles, e.g., CDC-EVs. In some embodiments, the lactate and glucose levels in the subject are decreased upon being treated with a therapeutically effective amount of extracellular vesicles, e.g., CDC-EVs. In some embodiments, the lactate and creatinine levels in the subject are decreased upon being treated with a therapeutically effective amount of extracellular vesicles, e.g., CDC-EVs. In some embodiments, the lactate, glucose and creatinine levels in the subject are decreased upon being treated with a therapeutically effective amount of extracellular vesicles, e.g., CDC-EVs. In some embodiments, said effective amount of extracellular vesicles, e.g., CDC-EVs, is administered to the subject immediately or within a short time period after coagulopathy, hemorrhagic shock and/or trauma. In some embodiments, said effective amount of extracellular vesicles, e.g., CDC-EVs, is administered to the subject within 1, 2, 3, 4, 5, 6, 7, 8, 9, 10, 11, 12, 13, 14, 15, 16, 17, 18, 19, 20, 21, 22, 23, or 24 hours after coagulopathy, hemorrhagic shock and/or trauma. In some embodiments, said therapeutically effective amount of CDC-EVs is administered to the subject systemically or locally. In some embodiments, said extracellular vesicles are exosomes, e.g., exosomes derived from cardiosphere-derived cells (CDC-derived exosomes). In some embodiments, are exosomes, microvesicles, membrane particles, membrane vesicles, exosome-like vesicles, ectosomes, ectosome-like vesicles, or exovesicles. In some embodiments, said subject is a mammalian subject, e.g., a human subject. In some embodiments, said trauma comprises one or more organ system failure, e.g., liver and/or kidney failure. In some embodiments, said formulation is suitable for use in combat casualty care. In some embodiments, said formulation is suitable for use as an anti-shock therapeutic.

The present invention provides, in a seventeenth aspect, a formulation comprising extracellular vesicles (EVs), e.g., EVs derived from cardiosphere-derived cells (CDC-EVs), for use in treating trauma to an organ in a subject in need thereof. In some embodiments, said organ is selected from the liver, kidneys, intestines, heart, arteries, veins, stomach, lungs, brain, spinal cord, cerebellum, nerves, placenta, uterus, spleen, bladder, esophagus, pancreas, colon, rectum, sex organs, pharynx, larynx, trachea, bronchi, salivary glands, diaphragm, skeletal muscles, bones, bone marrow, cartilage, ligaments, tendons, lymphatic vessels, ureters, urethra, endocrine glands, blood vessels, and gallbladder. In some embodiments, said organ is the liver. In some embodiments, said organ is one or both of the kidneys. In some embodiments, said organ is the liver and one or both of the kidneys. In some embodiments, said trauma is associated with, or is coupled with, or is induced by, hemorrhagic shock. In some embodiments, said trauma is associated with, or is coupled with, or is induced by, coagulopathy. In some embodiments, said trauma is associated with, or is coupled with, or is induced by, coagulopathy and hemorrhagic shock. In some embodiments, said trauma is polytrauma. In some embodiments, said trauma is acute trauma, e.g., acute polytrauma. In some embodiments, the lactate level in the subject is decreased upon being treated with a therapeutically effective amount of extracellular vesicles, e.g., CDC-EVs. In some embodiments, the lactate and glucose levels in the subject are decreased upon being treated with a therapeutically effective amount of extracellular vesicles, e.g., CDC-EVs. In some embodiments, the lactate and creatinine levels in the subject are decreased upon being treated with a therapeutically effective amount of extracellular vesicles, e.g., CDC-EVs. In some embodiments, the lactate, glucose and creatinine levels in the subject are decreased upon being treated with a therapeutically effective amount of extracellular vesicles, e.g., CDC-EVs. In some embodiments, said effective amount of extracellular vesicles, e.g., CDC-EVs, is administered to the subject immediately or within a short time period after coagulopathy, hemorrhagic shock and/or trauma. In some embodiments, said effective amount of extracellular vesicles, e.g., CDC-EVs, is administered to the subject within 1, 2, 3, 4, 5, 6, 7, 8, 9, 10, 11, 12, 13, 14, 15, 16, 17, 18, 19, 20, 21, 22, 23, or 24 hours after coagulopathy, hemorrhagic shock and/or trauma. In some embodiments, said therapeutically effective amount of CDC-EVs is administered to the subject systemically or locally. In some embodiments, said extracellular vesicles are exosomes, e.g., exosomes derived from cardiosphere-derived cells (CDC-derived exosomes). In some embodiments, are exosomes, microvesicles, membrane particles, membrane vesicles, exosome-like vesicles, ectosomes, ectosome-like vesicles, or exovesicles. In some embodiments, said subject is a mammalian subject, e.g., a human subject. In some embodiments, said formulation is suitable for use in combat casualty care. In some embodiments, said formulation is suitable for use as an anti-shock therapeutic.

In some embodiments of the aforementioned aspects of the present invention, said extracellular vesicles are derived from regenerative stem cells such as embryonic stem cells, pluripotent stem cells, multipotent stem cells, induced pluripotent stem cells, post-natal stem cells, adult stem cells, mesenchymal stem cells, hematopoietic stem cells, endothelial stem cells, epithelial stem cells, neural stem cells, cardiac stem cells including cardiac progenitor cells, bone marrow-derived stem cells, adipose-derived stem cells, hepatic stem cells, peripheral blood derived stem cells, cord blood-derived stem cells, or placental stem cells. In some embodiments, said extracellular vesicles are derived from cardiospheres, cardiosphere-derived cells (CDCs), immortalized CDCs (e.g., engineered high-potency exosomes derived from immortalized CDCs), 293F cells, or activated-specialized tissue-effector cells (ASTECs). In some embodiments, said extracellular vesicles are derived from cardiosphere-derived cells (CDC-EVs). In some embodiments, said extracellular vesicles are exosomes, microvesicles, membrane particles, membrane vesicles, exosome-like vesicles, ectosomes, ectosome-like vesicles, or exovesicles. In some embodiments, said extracellular vesicles are exosomes derived from CDCs. In some embodiments, said CDC-EVs, e.g., said exosomes derived from CDCs, comprise, or are enriched for, microRNAs miR-210 and miR-146a. In some embodiments, said CDC-EVs, e.g., said exosomes derived from CDCs, further comprise, or are enriched for, one or more additional microRNA fragments selected from the group consisting of miR-26a, miR27-a, let-7e, mir-19b, miR-125b, mir-27b, let-7a, miR-19a, let-7c, miR-140-3p, miR-125a-5p, miR-150, miR-155, let-7b, miR-24, miR-423-5p, miR-22, let-7f, and combinations thereof. In some embodiments, said CDC-EVs, e.g., said exosomes derived from CDCs, comprise, or are enriched for, EV-YF1 (SEQ ID NO: 1; 5′-ggcugguccg augguagugg guuaucagaa cuuauuaaca uuagugucac uaaagu-3′) or a fragment thereof, and/or EV-YF1-U16 (SEQ ID NO: 2; 5′-ggcugguccg augguuagug gguuaucaga acuuauuaac auuaguguca cuaaagu-3′) or a fragment thereof. In some embodiments, said CDC-EVs, e.g., said exosomes derived from CDCs, comprise, or are enriched for, miR-345.

In some embodiments of the aforementioned aspects of the present invention, the CDCs are treated to generate high-potency exosomes. Any suitable means for generating high-potency exosomes can be used. In some embodiments, the CDCs are treated with one or more agent that activates Wnt/β-catenin signaling. In some embodiments, an agent that activates Wnt/β-catenin signaling can repress or downregulate an inhibitor of Wnt/β-catenin signaling. In some embodiments, the agent that activates Wnt/β-catenin signaling is a GSK3β inhibitor. In some embodiments, the GSK3β inhibitor is 6-bromoindirubin-3′-oxime (BIO) or tideglusib (or combinations thereof). In some embodiments, an agent that activates Wnt/β-catenin signaling represses expression of an inhibitor of Wnt/β-catenin signaling. In some embodiments, the agent inhibits expression of Mest or Extl1. In some embodiments, the agent is a short hairpin (sh) RNA that targets Mest or Extl1. In some embodiments, the CDCs are transduced with an shRNA that targets MEST or Extl1.

In some embodiments of the aforementioned aspects of the present invention, the CDCs are immortalized. Advantageously, in several embodiments, immortalized CDCs can be passaged more times than their non-immortalized counterpart. In some embodiments, immortalized CDCs can be passaged 8 times or more, e.g., 9 times or more, 10 times or more 11 times or more, 12 times or more, 15 times or more, 18 times or more, 20 times or more, 25 times or more, 30 times or more, 40 times or more, including 50 times or more after the cardiosphere formation stage. Any suitable means of immortalizing CDCs may be used. In some embodiments, CDCs are immortalized by transduction with simian virus 40 large and small T antigen (SV40 T+t). In some embodiments, the CDCs are immortalized (e.g., by SV40 T+t transduction) and treated with an agent that activates Wnt/β-catenin signaling (e.g., shRNA that targets MEST or Ext11). Reference to immortalized CDC-derived exosomes herein include exosomes derived from immortalized CDCs that have been engineered to have high potency, e.g., by activating Wnt/β-catenin signaling in the CDCs. Suitable means of generating high-potency exosomes, immortalized CDCs, and exosomes derived therefrom, are described in, e.g., WO 2019/152409 and Ibrahim et al., Nat Biomed Eng. 2019 September; 3(9):695-705, which disclosures are incorporated herein by reference in their entirety. WO 2019/152409 and Ibrahim et al., Nat Biomed Eng. 2019 September; 3(9):695-705 also describe ASTECs and EVs, e.g., exosomes, derived from ASTECs (ASTEX). Non-limiting examples of ASTEX include EVs, e.g., exosomes, derived from immortalized CDCs with exogenously increased β-catenin level (e.g., imCDC^{sh-mest} cells), and EVs, e.g., exosomes derived from normal human dermal fibroblasts with exogenously increased β-catenin and gata4 (i.e., NHDF^{βcat/gata4}).

In some embodiments of the aforementioned aspects of the present invention, said extracellular vesicles are obtained from CDCs using a 1000 kDa-10 kDa process. In some embodiments, said 1000 kDa-10 kDa process comprises sequentially using a 1000 kDa filter for ultra-filtration, and then a 10 kDa for diafiltration. In some embodiments, said subject is a mammal, and preferably a human. In some embodiments, said therapeutically effective amount of extracellular vesicles are administered to the subject systemically or locally. In some embodiments, said therapeutically effective amount of extracellular vesicles are administered to the subject two or more times. In some embodiments, said extracellular vesicles are administered to the subject immediately after trauma and/or hemorrhagic shock, e.g., within 0-2 hours of injury, within 1 hour of injury, within 2 hours of injury, within 12 hours after injury, or within 24 hours of injury.

In a further aspect, provided herein is a method of treating shock subsequent to trauma and hemorrhage in a mammalian subject, comprising administering to the subject a therapeutically effective amount of extracellular vesicles derived from cardiosphere-derived cells (CDC-EVs). In an embodiment, the extracellular vesicles comprise exosomes. In another embodiment, the mammal is a human. In another embodiment, the trauma comprises polytrauma. In another embodiment, the trauma comprises organ trauma. In another embodiment, the organ comprises liver, kidney, heart, intestines, colon and/or brain. In another embodiment, the trauma further comprises trauma to the skeletal system. In another embodiment, the trauma results from gunshot or explosion. In another embodiment, the CDC-EVs are administered within about 1 hour, about 2 hours, about 3 hours, about 4 hours, 5 hours, 6 hours, about 7 hours, about 8 hours, about 9 hours, about 10 hours, about 11 hours or about 12 hours after the trauma. In another embodiment, the administration reduces risks of subject mortality compared with non-administration. In another embodiment, the administration reduces serum levels of one or more of lactate, glucose and creatinine. In another embodiment, the subject is a military personnel. In another embodiment, the military personnel sustained the trauma during combat.

### BRIEF DESCRIPTION OF THE DRAWINGS

FIGS. 1A-1C show size distribution (n=3) of CDC-EVs isolated from three samples (FIG. 1A) Donor 1, (FIG. 1B) Donor 2, and (FIG. 1C) Donor 3.

FIGS. 2A-2B show gating strategy and representative figures, wherein CDC-EVs were conjugated to Dynabeads to increase resolution for flow cytometry. (FIG. 2A) CDC-EVs formed five distinct flow groups following bead conjugation: unbound EVs (red) that were split into EVs less than 700 nm and greater than 700 nm, EVs bound to single Dynabeads (blue; termed “Single Beads”), EVs bound to two bead aggregates (orange; termed “Aggregate 1”), and EVs bound to three bead aggregates (green; termed “Aggregate 2”). (FIG. 2B) Representative results obtained after analyzing the various flow groups for tissue factor (PE-A) and phosphatidylserine (APC-A). Items in the top-left quadrant are positive for tissue factor, while items in the bottom-right quadrant are positive for phosphatidylserine. Items in the top-right quadrants are positive for both, while items in the bottom-left quadrant are negative for both.

FIGS. 3A-3B show unpaired t-test analysis (n=3) of (FIG. 3A) phosphatidylserine (PS) and (FIG. 3B) tissue factor (TF) expression on CDC-EV surfaces. Graphs are depicted with individual data points alongside the mean. **** p<0.0001.

FIGS. 4A-4D show one-way ANOVA analysis (n=3) of CAT data. (FIG. 4A) Peak thrombin activity, (FIG. 4B) time to peak thrombin activity, (FIG. 4C) time to thrombin generation (lag time), and (FIG. 4D) endogenous thrombin potential (total amount of thrombin generated). Graphs are depicted with individual data points alongside the mean. *p<0.05, **p<0.01, ***p<0.001, ****p<0.0001.

FIG. 5 shows CDC-EV clotting capabilities as measured by thromboelastogram (TEG; values rounded to three significant figures). CDC-EVs (10 μg total protein) were mixed with platelet poor plasma to total 340 μL of sample. 20 μL of 0.2 M calcium chloride was then added to initiate clot formation. The following TEG parameters were measured:
SP-time (min): time necessary to detect the first fibrin of the clot
R-time (min): time for the clot to reach 2 mm; evaluates the activity of the coagulant factors
K-time (min): time necessary for the clot to reach 20 mm; a decreased K value reflects increased clotting kinetics
Angle (degrees): reflects the rate of clotting; a steep angle indicates a rapid response
MA (mm): the maximum amplitude of the clot size; corresponds to ultimate clot strength
G (dyne/cm²): reflection of clot strength

FIGS. 6A-6C show representative images of the effects of EVs or vehicle on platelet-collagen interaction. One-way ANOVA analysis of (FIG. 6B) peak platelet adhesion (percent surface coverage) and (FIG. 6C) peak platelet fluorescence intensity (FIU) during flow-based adhesion (n=3). Graphs are depicted with individual data points alongside the mean.

FIG. 7 shows that nine weeks old C57BL10 female mice were injected in the jugular vein with 0.1 mL of PlasmaLyte A (n=4) or with 1e10 particles in 0.1 mL of PlasmaLyte A (n=4) and sacrificed 1 h post administration. Lung, spleen, liver, brain, kidney and heart were collected and preserved in RNA later at −20° C. RNA was isolated from the different organs, and qPCR was performed for a human-specific RNA fragment, hY4f, using specific primers. Fold change versus control mice is shown. Significance was determined using student's t-test. *p<0.05 and **p<Graphs are depicted with individual data points alongside the mean.

FIGS. 8A-8B that rats were sacrificed 3 h after injection of CDC-EVs or vehicle into the femoral vein following trauma. RNA was isolated from (FIG. 8A) kidney and (FIG. 8B) liver tissue, and qPCR was performed for a human-specific RNA fragment, hY4f, which is abundant in CDC-EVs. Fold change was calculated using 2^{-ΔΔct} by comparing the ΔCt of each tissue to the ΔCt of that tissue from the PlasmaLyte-injected mice. Graphs are depicted with individual data points alongside the mean.

FIGS. 9A-9D show repeated measures of two-way ANOVA analysis of CDC-EV effects on rat (FIG. 9A) prothrombin times, (FIG. 9B) lactate levels, (FIG. 9C) glucose levels, and (FIG. 9D) creatinine levels following polytrauma and hemorrhage. Sham denotes rats treated with CDC-EVs but no trauma (n=4), vehicle denotes rats treated with Plasmalyte-A post-trauma (n=8), and EVs denotes rats treated with CDC-EVs post-trauma (n=8). *p<0.05, **p<0.01, ***p<0.001, and ****p<0.0001.

FIG. 10 shows CDC-EVs were processed to probe for EV specific markers at the protein level (western blot; WB) and at the gene level (RT-qPCR), and to confirm morphology using transmission electron microscopy (TEM). HEK293 cell-derived EVs were used as a positive control. (FIG. 10A) Immunoblot analysis of CDC-EVs was performed to detect the protein expression of an exosome specific marker, CD9. EV enrichment was confirmed for the expression of the specific exosomal marker CD9 in CDC-EVs and HEK293-EVs that served as positive control. (FIG. 10B) EVs were assessed at gene level by RT-qPCR analysis for the expression of CD63, a marker that is present in both microvesicles and exosomes (components of EVs). GAPDH was used as an internal housekeeping gene.

### DETAILED DESCRIPTION

### A. Definitions

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

As used herein, “and/or” refers to and encompasses any and all possible combinations of one or more of the associated listed items, as well as the lack of combinations when interpreted in the alternative (or).

The terms “about” and “approximate”, as used herein when referring to a measurable value such as an amount, dose, time, temperature, activity, level, number, frequency, percentage, dimension, size, amount, weight, position, length and the like, is meant to encompass variations of ±15%, ±10%, ±5%, ±1%, ±0.5%, or even ±0.1% of the specified amount, dose, time, temperature, activity, level, number, frequency, percentage, dimension, size, amount, weight, position, length and the like. In instances in which the terms “about” and “approximate” are used in connection with the location or position of regions within a reference polypeptide, these terms encompass variations of ±up to 20 amino acid residues, ±up to 15 amino acid residues, ±up to 10 amino acid residues, ±up to 5 amino acid residues, ±up to 4 amino acid residues, ±up to 3 amino acid residues, ±up to 2 amino acid residues, or even ±1 amino acid residue.

As used herein, the following meanings apply unless otherwise specified. The word “may” is used in a permissive sense (i.e., meaning having the potential to), rather than the mandatory sense (i.e., meaning must). The words “include”, “including”, and “includes” and the like mean including, but not limited to.

The term “derived from” as in “A is derived from B” means that A is obtained from B in such a manner that A is not identical to B.

The terms “therapy,” “treatment,” and “amelioration” refer to any reduction in the severity of symptoms. The terms “treat” and “prevent” are not intended to be absolute terms. Treatment and prevention can refer to any delay in onset, amelioration of symptoms, improvement in patient survival, increase in survival time or rate, etc. Treatment and prevention can be complete or partial. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

A treatment can be considered “effective,” as used herein, if one or more of the signs or symptoms of a condition described herein are altered in a beneficial manner, other clinically accepted symptoms are improved, or even ameliorated, or a desired response is induced e.g., by at least 2%, 3%, 4%, 5%, 10%, or more, following treatment according to the methods described herein. Efficacy can be assessed, for example, by measuring a marker, indicator, symptom, and/or the incidence of a condition treated according to the methods described herein or any other measurable parameter appropriate. Efficacy can also be measured by a failure of an individual to worsen as assessed by hospitalization, or need for medical interventions (e.g., progression of the disease is halted). Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. One skilled in the art can monitor efficacy of administration and/or treatment by measuring any one of such parameters, or any combination of parameters.

The term “effective amount” as used herein refers to the amount of a composition or an agent needed to alleviate at least one or more symptom of the disease or disorder, and relates to a sufficient amount of therapeutic composition to provide the desired effect. The term “therapeutically effective amount” refers to an amount of a composition or therapeutic agent that is sufficient to provide a particular effect when administered to a typical subject. An effective amount as used herein, in various contexts, can include an amount sufficient to delay the development of a symptom of the disease, alter the course of a symptom disease (for example but not limited to, slowing the progression of a symptom of the disease), or reverse a symptom of the disease. For example, for the given parameter, a therapeutically effective amount will show an increase or decrease of therapeutic effect at least any of 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least any of a 1.2-fold, 1.5-fold, 2-fold, 5-fold, or more effect over a control. The therapeutically effective amount may be administered in one or more doses of the therapeutic agent. The therapeutically effective amount may be administered in a single administration, or over a period of time in a plurality of doses.

“Administering” as used herein can include any suitable routes of administering a therapeutic agent or composition as disclosed herein. Suitable routes of administration include, without limitation, oral, parenteral, intravenous, intramuscular, subcutaneous, transdermal, airway (aerosol), pulmonary, cutaneous, injection or topical administration. Administration can be local or systemic.

The term “pharmaceutical composition” refers to a formulation comprising an active ingredient, and optionally a pharmaceutically acceptable carrier, diluent or excipient. The term “active ingredient” can interchangeably refer to an “effective ingredient,” and is meant to refer to any agent that is capable of inducing a sought-after effect upon administration. By “pharmaceutically acceptable” it is meant the carrier, diluent or excipient must be compatible with the other ingredients of the formulation and not deleterious to the recipient thereof, nor to the activity of the active ingredient of the formulation. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG). Examples of carrier include, but are not limited to, liposome, nanoparticles, ointment, micelles, microsphere, microparticle, cream, emulsion, and gel. Examples of excipient include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluent include, but are not limited to, water, alcohol, saline solution, glycol, mineral oil and dimethyl sulfoxide (DMSO).

The terms “dose” and “dosage” are used interchangeably herein. A dose refers to the amount of active ingredient given to an individual at each administration. For the present invention, the dose can refer to the concentration of the extracellular vesicles or associated components, e.g., the amount of therapeutic agent or dosage of radiolabel. The dose will vary depending on a number of factors, including frequency of administration; size and tolerance of the individual; severity of the condition; risk of side effects; the route of administration; and the imaging modality of the detectable moiety (if present). One of skill in the art will recognize that the dose can be modified depending on the above factors or based on therapeutic progress. The term “dosage form” refers to the particular format of the pharmaceutical, and depends on the route of administration. For example, a dosage form can be in a liquid, e.g., a saline solution for injection.

“Subject,” “patient,” “individual” and like terms are used interchangeably and refer to, except where indicated, mammals such as humans and non-human primates, as well as rabbits, rats, mice, goats, pigs, and other mammalian species. The term does not necessarily indicate that the subject has been diagnosed with a particular disease, but typically refers to an individual under medical supervision. A patient can be an individual that is seeking treatment, monitoring, adjustment or modification of an existing therapeutic regimen, etc.

The term “extracellular vesicle” (EV) refers to lipid bilayer-delimited particles that are naturally released from cells. EVs range in diameter from around 20-30 nanometers to about 10 microns or more. EVs can comprise proteins, nucleic acids, lipids and metabolites from the cells that produced them. EVs include exosomes (about 50 to about 100 nm), microvesicles (about 100 to about 300 nm), ectosomes (about 50 to about 1000 nm), apoptotic bodies (about 50 to about 5000 nm) and lipid-protein aggregates of the same dimensions.

The term “trauma” or “traumatic” has its ordinary meaning as understood by one of ordinary skill in the art and in view of the present disclosure. For instance, traumatic injury may include direct or indirect physical (e.g., mechanical) damage to a subject's body part caused by a physical impact, and it may involve single or multiple organ system failure. Trauma includes blunt trauma and penetrating injuries. The physical impact may be any form of physical interaction with the body that causes tissue damage, including those caused by mechanical, thermal, electromagnetic, and acoustic impact to the tissue. A traumatic injury can be caused suddenly by a single impact or a series of physical impacts that occurs within a short period of time. A traumatic injury can cause physical loss of tissue and/or single or multiple organ system failure. Traumatic injury can be severe enough that the body's natural repair mechanisms (e.g., regeneration of tissue without medical intervention directed to promoting such tissue regeneration) is inadequate to restore some or all function of the injured tissue and/or organ system. A traumatic injury may be caused accidentally, inadvertently, or intentionally. The term “polytrauma” refers to trauma, i.e., injuries, to a plurality of body parts and/or organ systems. “Polytrauma associated with, coupled with, or induced by, hemorrhagic shock” is defined as circulatory dysfunction leading to the accumulation of oxygen debt and multiple organ system failure.

“Acute” conditions are severe and sudden in onset. This could describe anything from a broken bone to an asthma attack. A chronic condition, by contrast is a long-developing syndrome, such as osteoporosis or asthma. The term “acute” often also connotes an illness that is of short duration, rapidly progressive, and in need of urgent care.

The term “coagulopathy” has its ordinary meaning as understood by one of ordinary skill in the art and in view of the present disclosure. Coagulopathy is a condition in which the blood's ability to coagulate, or form clots, is impaired. This condition can cause a tendency toward prolonged or excessive bleeding, which may occur spontaneously or following an injury, e.g., traumatic injury, or medical and dental procedures. Coagulopathy may cause uncontrolled internal or external bleeding. Left untreated, uncontrolled bleeding may cause damage to joints, muscles, or internal organs and may be life-threatening. coagulopathy induced by trauma may results in more severe bleeding and/or multi-organ failure.

The term “trauma-induced coagulopathy” means coagulopathy resulting from traumatic injury.

The term “hemorrhagic shock” has its ordinary meaning as understood by one of ordinary skill in the art and in view of the present disclosure. Hemorrhagic shock occurs when the body begins to shut down due to large amounts of blood loss. Hemorrhagic shock is a clinical syndrome resulting from decreased blood volume (hypovolemia) caused by blood loss, which leads to reduced cardiac output and organ perfusion. Blood loss can be external (e.g., externally bleeding wound) or internal (e.g., internal bleeding caused by ruptured aortic aneurism). The severity of hemorrhagic shock and associated symptoms depends on the volume of blood that is lost and how rapidly it is lost.

Definitions of common terms in cell biology and molecular biology can be found in “The Merck Manual of Diagnosis and Therapy”, 19th Edition, published by Merck Research Laboratories, 2006 (ISBN 0-91 1910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

### B. Cardiospheres

Cardiospheres are undifferentiated cardiac cells that grow as self-adherent clusters as described in WO 2005/012510, and Messina et al., “Isolation and Expansion of Adult Cardiac Stem Cells from Human and Murine Heart,” Circulation Research, 95:911-921 (2004), the disclosures of which are herein incorporated by reference in their entirety.

Briefly, heart tissue can be collected from a patient during surgery or cardiac biopsy. The heart tissue can be harvested from the left ventricle, right ventricle, septum, left atrium, right atrium, crista terminalis, right ventricular endocardium, septal or ventricle wall, atrial appendages, or combinations thereof. A biopsy can be obtained, e.g., by using a percutaneous bioptome as described in, e.g., U.S. Patent Application Publication Nos. 2009/012422 and 2012/0039857, the disclosures of which are herein incorporated by reference in their entirety. The tissue can then be cultured directly, or alternatively, the heart tissue can be frozen, thawed, and then cultured. The tissue can be digested with protease enzymes such as collagenase, trypsin and the like. The heart tissue can be cultured as an explant such that cells including fibroblast-like cells and cardiosphere-forming cells grow out from the explant. In some instances, an explant is cultured on a culture vessel coated with one or more components of the extracellular matrix (e.g., fibronectin, laminin, collagen, elastin, or other extracellular matrix proteins). The tissue explant can be cultured for about 1, 2, 3, 4, or more weeks prior to collecting the cardiosphere-forming cells. A layer of fibroblast-like cells can grow from the explant onto which cardiosphere-forming cells appear. Cardiosphere-forming cells can appear as small, round, phase-bright cells under phase contrast microscopy. Cells surrounding the explant including cardiosphere-forming cells can be collected by manual methods or by enzymatic digestion. The collected cardiosphere-forming cells can be cultured under conditions to promote the formation of cardiospheres. In some aspects, the cells are cultured in cardiosphere-growth medium comprising buffered media, amino acids, nutrients, serum or serum replacement, growth factors including but not limited to EGF and bFGF, cytokines including but not limited to cardiotrophin, and other cardiosphere promoting factors such as but not limited to thrombin. Cardiosphere-forming cells can be plated at an appropriate density necessary for cardiosphere formation, such as about 20,000-100,000 cells/mL. The cells can be cultured on sterile dishes coated with poly-D-lysine, or other natural or synthetic molecules that hinder the cells from attaching to the surface of the dish. Cardiospheres can appear spontaneously about 2-7 days or more after cardiosphere-forming cells are plated.

### C. Cardiosphere-Derived Cells (CDCs)

CDCs are a population of cells generated by manipulating cardiospheres in the manner as described in, e.g., U.S. Patent Application Publication No. 2012/0315252, the disclosures of which are herein incorporated by reference in their entirety. For example, CDCs can be generated by plating cardiospheres on a solid surface which is coated with a substance which encourages adherence of cells to a solid surface of a culture vessel, e.g., fibronectin, a hydrogel, a polymer, laminin, serum, collagen, gelatin, or poly-D-lysine, and expanding same as an adherent monolayer culture. CDCs can be repeatedly passaged, e.g., passaged two times or more, according to standard cell culturing methods.

### D. Activated-Specialized Tissue-Effector Cells (ASTECs) and ASTEC-Derived Exosomes (ASTEX)

ASTECs and ASTEX are activation-specialized tissue-effector cells and EVs, e.g, exosomes, derived therefrom, respectively, as described in WO 2019/152409, and Ibrahim et al., Augmenting canonical Wnt signalling in therapeutically inert cells converts them into therapeutically potent exosome factories, Nat Biomed Eng., 2019 September; 3(9):695-705, the disclosures of which are herein incorporated by reference in their entirety.

### E. Exosomes

Exosomes are vesicles formed via a specific intracellular pathway involving multivesicular bodies or endosomal-related regions of the plasma membrane of a cell. Exosomes can range in size from approximately 20-150 nm in diameter. In some cases, they have a characteristic buoyant density of approximately 1.1-1.2 g/mL, and a characteristic lipid composition. Their lipid membrane is typically rich in cholesterol and contains sphingomyelin, ceramide, lipid rafts and exposed phosphatidylserine. Exosomes express certain marker proteins, such as integrins and cell adhesion molecules, but generally lack markers of lysosomes, mitochondria, or caveolae. In some embodiments, the exosomes contain cell-derived components, such as but not limited to, proteins, DNA and RNA (e.g., microRNA and noncoding RNA). In some embodiments, exosomes can be obtained from cells obtained from a source that is allogeneic, autologous, xenogeneic, or syngeneic with respect to the recipient of the exosomes.

Certain types of RNA, e.g., microRNA (miRNA), are known to be carried by exosomes. miRNAs function as post-transcriptional regulators, often through binding to complementary sequences on target messenger RNA transcripts (mRNAs), thereby resulting in translational repression, target mRNA degradation and/or gene silencing. For example, as described in WO/2014/028493, miR146a exhibits over a 250-fold increased expression in CDCs, and miR210 is upregulated approximately 30-fold, as compared to the exosomes isolated from normal human dermal fibroblasts.

Exosomes derived from cardiospheres and CDCs are described in, e.g., WO/2014/028493, the disclosures of which are herein incorporated by reference in their entirety. Methods for preparing exosomes can include the steps of: culturing cardiospheres or CDCs in conditioned media, isolating the cells from the conditioned media, purifying the exosome by, e.g., sequential centrifugation, and optionally, clarifying the exosomes on a density gradient, e.g., sucrose density gradient. In some instances, the isolated and purified exosomes are essentially free of non-exosome components, such as components of cardiospheres or CDCs. Exosomes can be resuspended in a buffer such as a sterile PBS buffer containing 0.01-1% human serum albumin. The exosomes may be frozen and stored for future use.

Exosomes can be prepared using a commercial kit such as, but not limited to the ExoSpin™ Exosome Purification Kit, Invitrogen® Total Exosome Purification Kit, PureExo® Exosome Isolation Kit, and ExoCap™ Exosome Isolation kit. Methods for isolating exosome from stem cells are found in, e.g., Tan et al., Journal of Extracellular Vesicles, 2:22614 (2013); Ono et al., Sci Signal, 7(332):ra63 (2014) and U.S. Application Publication Nos. 2012/0093885 and 2014/0004601. Methods for isolating exosome from cardiosphere-derived cells are found in, e.g., Ibrahim et al., Exosomes as critical agents of cardiac regeneration triggered by cell therapy, Stem Cell Reports, 2014. Collected exosomes can be concentrated and/or purified using methods known in the art. Specific methodologies include ultracentrifugation, density gradient, HPLC, adherence to substrate based on affinity, or filtration based on size exclusion.

For example, differential ultracentrifugation has become a leading technique wherein secreted exosomes are isolated from the supernatants of cultured cells. This approach allows for separation of exosomes from nonmembranous particles, by exploiting their relatively low buoyant density. Size exclusion allows for their separation from biochemically similar, but biophysically different microvesicles, which possess larger diameters of up to 1,000 nm. Differences in flotation velocity further allows for separation of differentially sized exosomes. In general, exosome sizes will possess a diameter ranging from 30-200 nm, including sizes of 40-100 nm. Further purification may rely on specific properties of the particular exosomes of interest. This includes, e.g., use of immunoadsorption with a protein of interest to select specific vesicles with exoplasmic or outward orientations.

Among current methods, e.g., differential centrifugation, discontinuous density gradients, immunoaffinity, ultrafiltration and high performance liquid chromatography (HPLC), differential ultracentrifugation is the most commonly used for exosome isolation. This technique utilizes increasing centrifugal force from 2000×g to 10,000×g to separate the medium- and larger-sized particles and cell debris from the exosome pellet at 100,000×g. Centrifugation alone allows for significant separation/collection of exosomes from a conditioned medium, although it is insufficient to remove various protein aggregates, genetic materials, particulates from media and cell debris that are common contaminants. Enhanced specificity of exosome purification may deploy sequential centrifugation in combination with ultrafiltration, or equilibrium density gradient centrifugation in a sucrose density gradient, to provide for the greater purity of the exosome preparation (flotation density 1.1-1.2 g/mL) or application of a discrete sugar cushion in preparation.

Importantly, ultrafiltration can be used to purify exosomes without compromising their biological activity. Membranes with different pore sizes—such as 100 kDa molecular weight cutoff (MWCO) and gel filtration to eliminate smaller particles—have been used to avoid the use of a nonneutral pH or non-physiological salt concentration. Currently available tangential flow filtration (TFF) systems are scalable (to >10,000 L), allowing one to not only purify, but concentrate the exosome fractions, and such approaches are less time consuming than differential centrifugation. HPLC can also be used to purify exosomes to homogeneouslysized particles and preserve their biological activity as the preparation is maintained at a physiological pH and salt concentration.

Other chemical methods have exploit differential solubility of exosomes for precipitation techniques, addition to volume-excluding polymers (e.g., polyethylene glycols (PEGs)), possibly combined additional rounds of centrifugation or filtration. For example, a precipitation reagent, ExoQuick®, can be added to conditioned cell media to quickly and rapidly precipitate a population of exosomes, although re-suspension of pellets prepared via this technique may be difficult. Flow field-flow fractionation (FlFFF) is an elution-based technique that is used to separate and characterize macromolecules (e.g., proteins) and nano- to micro-sized particles (e.g., organelles and cells) and which has been successfully applied to fractionate exosomes from culture media.

Beyond these techniques relying on general biochemical and biophysical features, focused techniques may be applied to isolate specific exosomes of interest. This includes relying on antibody immunoaffinity to recognizing certain exosome-associated antigens. As described, exosomes further express the extracellular domain of membrane-bound receptors at the surface of the membrane. This presents a ripe opportunity for isolating and segregating exosomes in connections with their parental cellular origin, based on a shared antigenic profile. Conjugation to magnetic beads, chromatography matrices, plates or microfluidic devices allows isolating of specific exosome populations of interest as may be related to their production from a parent cell of interest or associated cellular regulatory state. Other affinity-capture methods use lectins which bind to specific saccharide residues on the exosome surface.

In several embodiments, there are provided compositions comprising exosomes (e.g., exosomes derived from CDCs) for use in repair or regeneration of tissues that have been adversely impacted by damage or disease. In several embodiments, the compositions comprise, consist of, or consist essentially of exosomes. In some embodiments, the exosomes comprise nucleic acids, proteins, or combinations thereof. In several embodiments, the nucleic acids within the exosomes comprise one or more types of RNA (though certain embodiments involved exosomes comprising DNA). The RNA, in several embodiments, comprises one or more of messenger RNA, snRNA, saRNA, miRNA, and combinations thereof. In several embodiments, the miRNA comprises one or more of miR-92a, miR-26a, miR27-a, let-7e, miR-19b, miR-125b, mir-27b, let-7a, miR-19a, let-7c, miR-140-3p, miR-125a-5p, miR-150, miR-155, miR-210, let-7b, miR-24, miR-423-5p, miR-22, let-7f, miR-146a, and combinations thereof. In several embodiments, the compositions comprise, consist of, or consist essentially of a synthetic microRNA and a pharmaceutically acceptable carrier. In some such embodiments, the synthetic microRNA comprises miR146a. In several embodiments the miRNA is pre-miRNA (e.g., not mature), while in some embodiments, the miRNA is mature, and in still additional embodiments, combinations of pre-miRNA and mature miRNA are used.

In several embodiments, the compositions comprise exosomes derived from a population of cells, as well as one or more cells from the population (e.g., a combination of exosomes and their “parent cells”). In several embodiments, the compositions comprise a plurality of exosomes derived from a variety of cell types (e.g., a population of exosomes derived from a first and a second type of “parent cell”). As discussed above, in several embodiments, the compositions disclosed herein may be used alone, or in conjunction with one or more adjunct therapeutic modalities (e.g., pharmaceutical, cell therapy, gene therapy, protein therapy, surgery, etc.).

In several embodiments, the exosomes are about 15 nm to about 95 nm in diameter, including about 15 nm to about 20 nm, about 20 nm to about 25 nm, about 25 nm to about 30 nm, about 30 nm to about 35 nm, about 35 nm to about 40 nm, about 40 nm to about 50 nm, about 50 nm to about 60 nm, about 60 nm to about 70 nm, about 70 nm to about 80 nm, about 80 nm to about 90 nm, about 90 nm to about 95 nm and overlapping ranges thereof. In certain embodiments, larger EVs are obtained that are larger in diameter (e.g., those ranging from about 140 to about 210 nm). Advantageously, in several embodiments, the exosomes comprise synthetic membrane bound particles (e.g., exosome surrogates), which depending on the embodiment, are configured to a specific range of diameters. In such embodiments, the diameter of the exosome surrogates is tailored for a particular application (e.g., target site or route of delivery). In still additional embodiments, the exosome surrogates are labeled or modified to enhance trafficking to a particular site or region post-administration.

In several embodiments, exosomes are obtained via centrifugation of the regenerative cells. In several embodiments, ultracentrifugation is used. However, in several embodiments, ultracentrifugation is not used. In several embodiments, exosomes are obtained via size-exclusion filtration of the regenerative cells. As disclosed above, in some embodiments, synthetic exosomes are generated, which can be isolated by similar mechanisms as those above.

In several embodiments, the exosomes induce altered gene expression by repressing translation and/or cleaving mRNA, for example. In some embodiments, the alteration of gene expression results in inhibition of undesired proteins or other molecules, such as those that are involved in cell death pathways, or induce further damage to surrounding cells (e.g., free radicals). In several embodiments, the alteration of gene expression results directly or indirectly in the creation of desired proteins or molecules (e.g., those that have a beneficial effect). The proteins or molecules themselves need not be desirable per se (e.g., the protein or molecule may have an overall beneficial effect in the context of the damage to the tissue, but in other contexts would not yield beneficial effects). In some embodiments, the alteration in gene expression causes repression of an undesired protein, molecule or pathway (e.g., inhibition of a deleterious pathway). In several embodiments, the alteration of gene expression reduces the expression of one or more inflammatory agents and/or the sensitivity to such agents. Advantageously, the administration of exosomes, or miRNAs, in several embodiments, results in downregulation of certain inflammatory molecules and/or molecules involved in inflammatory pathways. As such, in several embodiments, cells that are contacted with the exosomes or miRNAs enjoy enhanced viability, even in the event of post-injury inflammation or inflammation due to disease.

In several embodiments, the exosomes fuse with one or more recipient cells of the damaged tissue. In several embodiments, the exosomes release the microRNA into one or more recipient cells of the damaged tissue, thereby altering at least one pathway in the one or more cells of the damaged tissue. In some embodiments, the exosomes exerts their influence on cells of the damaged tissue by altering the environment surrounding the cells of the damaged tissue. In some embodiments, signals generated by or as a result of the content or characteristics of the exosomes, lead to increases or decreases in certain cellular pathways. For example, the exosomes (or their contents/characteristics) can alter the cellular milieu by changing the protein and/or lipid profile, which can, in turn, lead to alterations in cellular behavior in this environment. Additionally, in several embodiments, the miRNA of an exosome can alter gene expression in a recipient cell, which alters the pathway in which that gene was involved, which can then further alter the cellular environment. In several embodiments, the influence of the exosomes directly or indirectly stimulates angiogenesis. In several embodiments, the influence of the exosomes directly or indirectly affects cellular replication. In several embodiments, the influence of the exosomes directly or indirectly inhibits cellular apoptosis.

The beneficial effects of the exosomes (or their contents) need not only be on directly damaged or injured cells. In some embodiments, for example, the cells of the damaged tissue that are influenced by the disclosed methods are healthy cells. However, in several embodiments, the cells of the damaged tissue that are influenced by the disclosed methods are damaged cells.

In several embodiments, the regenerative cells and/or exosomes are mammalian in origin. In several embodiments, the regenerative cells and/or exosomes are human in origin. In some embodiments, the cells and/or exosomes are non-embryonic human regenerative cells and/or exosomes. In several embodiments, the regenerative cells and/or exosomes are autologous to the individual while in several other embodiments the regenerative cells and/or exosomes are allogeneic to the individual. Xenogeneic or syngeneic cells and/or exosomes are used in certain other embodiments.

Upon administration (discussed in more detail below) exosomes can fuse with the cells of a target tissue. As used herein, the term “fuse” shall be given its ordinary meaning and shall also refer to complete or partial joining, merging, integration, or assimilation of the exosome and a target cell. In several embodiments, the exosomes fuse with healthy cells of a target tissue. In some embodiments, the fusion with healthy cells results in alterations in the healthy cells that leads to beneficial effects on the damaged or diseased cells (e.g., alterations in the cellular or intercellular environment around the damaged or diseased cells). In some embodiments, the exosomes fuse with damaged or diseased cells. In some such embodiments, there is a direct effect on the activity, metabolism, viability, or function of the damaged or diseased cells that results in an overall beneficial effect on the tissue. In several embodiments, fusion of the exosomes with either healthy or damaged cells is not necessary for beneficial effects to the tissue as a whole (e.g., in some embodiments, the exosomes affect the intercellular environment around the cells of the target tissue). Thus, in several embodiments, fusion of the exosome to another cell does not occur. In several embodiments, there is no cell-exosome contact, yet the exosomes still influence the recipient cells.

In several embodiments, administration of exosomes is particularly advantageous because there are reduced complications due to immune rejection by the recipient. Certain types of cellular or gene therapies are hampered by the possible immune response of a recipient of the therapy. As with organ transplants or tissue grafts, certain types of foreign cells (e.g., not from the recipient) are attacked and eliminated (or rendered partially or completely non-functional) by recipient immune function. One approach to overcome this is to co-administer immunosuppressive therapy, however this can be costly, and leads to a patient being subject to other infectious agents. Thus, exosomal therapy is particularly beneficial because the immune response is limited. In several embodiments, this allows the use of exosomes derived from allogeneic cell sources (though in several embodiments, autologous sources are used). Moreover, the reduced potential for immune response allows exosomal therapy to be employed in a wider patient population, including those that are immunocompromised and those that have hyperactive immune systems. Moreover, in several embodiments, because the exosomes do not carry a full complement of genetic material, there is a reduced risk of unwanted cellular growth (e.g., teratoma formation) post-administration. In several embodiments, in order to further reduce the risk of recipient immune response and/or teratoma formation, exosomes can be further manipulated, for example through gene editing using, for example CRISPR-Cas, zinc finger nucleases, and/or TALENs, to reduce their potential immunogenicity. Advantageously, the exosomes can be derived, depending on the embodiment, from cells obtained from a source that is allogeneic, autologous, xenogeneic, or syngeneic with respect to the eventual recipient of the exosomes. Moreover, master banks of exosomes that have been characterized for their expression of certain miRNAs and/or proteins can be generated and stored long-term for subsequent use in defined subjects on an “off-the-shelf” basis. However, in several embodiments, exosomes are isolated and then used without long-term or short-term storage (e.g., they are used as soon as practicable after their generation).

In several embodiments, exosomes are administered in combination with one or more additional agents. For example, in several embodiments, the exosomes are administered in combination with one or more proteins or nucleic acids derived from the exosome (e.g., to supplement the exosomal contents). In several embodiments, the cells from which the exosomes are isolated are administered in conjunction with the exosomes. In several embodiments, such an approach advantageously provides an acute and more prolonged duration of exosome delivery (e.g., acute based on the actual exosome delivery and prolonged based on the cellular delivery, the cells continuing to secrete exosomes post-delivery).

In several embodiments, exosomes are delivered in conjunction with a more traditional therapy, e.g., surgical therapy or pharmaceutical therapy. In several embodiments such combinations of approaches result in synergistic improvements in the viability and/or function of the target tissue. In some embodiments, exosomes may be delivered in conjunction with a gene therapy vector (or vectors), nucleic acids (e.g., those used as siRNA or to accomplish RNA interference), and/or combinations of exosomes derived from other cell types.

### F. 10 KDa & 1000 KDa Method

CDC-EV (10 KDa or 1000 KDa) drug substance is obtained after filtering CDC conditioned medium (CM) containing EVs through a 10 KDa or 1000 KDa pore size filter, wherein the final product, composed of secreted EVs and concentrated CM, is formulated in PlasmaLyte A by diafiltration and stored frozen.

### G. MSC-EVs

EVs originating from human bone marrow mesenchymal stem cells (MSC-EVs) are obtained after filtering MSC CM containing EVs through a 10 KDa pore size filter following a similar process as for CDC-EV production. MSC-EVs are a non-cellular, filter sterilized product obtained from human MSCs cultured under defined, serum-free conditions. The final product, composed of secreted EVs and concentrated CM, is formulated in PlasmaLyte A and stored frozen. The frozen final product is “ready to use” for direct subconjunctival injection after thawing.

### H. Administration

The compositions disclosed herein can be administered by one of many routes, depending on the embodiment. For example, exosome administration may be by local or systemic administration. Local administration, depending on the tissue to be treated, may in some embodiments be achieved by direct administration to a tissue (e.g., direct injection, such as intramyocardial injection). Local administration may also be achieved by, for example, lavage of a particular tissue (e.g., intra-intestinal or peritoneal lavage). In several embodiments, systemic administration is used and may be achieved by, for example, intravenous and/or intraarterial delivery. In certain embodiments, intracoronary delivery is used. In several embodiments, the exosomes are specifically targeted to the damaged or diseased tissues. In some such embodiments, the exosomes are modified (e.g., genetically or otherwise) to direct them to a specific target site. For example, modification may, in some embodiments, comprise inducing expression of a specific cell-surface marker on the exosome, which results in specific interaction with a receptor on a desired target tissue. In one embodiment, the native contents of the exosome are removed and replaced with desired exogenous proteins or nucleic acids. In one embodiment, the native contents of exosomes are supplemented with desired exogenous proteins or nucleic acids. In some embodiments, however, targeting of the exosomes is not performed. In several embodiments, exosomes are modified to express specific nucleic acids or proteins, which can be used, among other things, for targeting, purification, tracking, etc. In several embodiments, however, modification of the exosomes is not performed. In some embodiments, the exosomes do not comprise chimeric molecules.

In some embodiments, subcutaneous or transcutaneous delivery methods are used. Due to the relatively small size, exosomes are particularly advantageous for certain types of therapy because they can pass through blood vessels down to the size of the microvasculature, thereby allowing for significant penetration into a tissue. In some embodiments, this allows for delivery of the exosomes directly to central portion of the damaged or diseased tissue. In addition, in several embodiments, use of exosomes is particularly advantageous because the exosomes can deliver their payload (e.g., the resident nucleic acids and/or proteins) across the blood brain barrier, which has historically presented an obstacle to many central nervous system therapies. In certain embodiments, however, exosomes may be delivered to the central nervous system by injection through the blood brain barrier. In several embodiments, exosomes are particularly beneficial for administration because they permit lower profile delivery devices for administration (e.g., smaller size catheters and/or needles). In several embodiments, the smaller size of exosomes enables their navigation through smaller and/or more convoluted portions of the vasculature, which in turn allows exosomes to be delivered to a greater portion of most target tissues.

The dose of exosomes administered, depending on the embodiment, ranges from about 1.0×10⁵ to about 1.0×10⁹ exosomes, including about 1.0×10⁵ to about 1.0×10⁶, about 1.0×10⁶ to about 1.0×10⁷, about 1.0×10⁷ to about 5.0×10⁷, about 5.0×10⁷ to about 1.0×10⁸, about 1.0×10⁸ to about 2.0×10⁸, about 2.0×10⁸ to about 3.5×10⁸, about 3.5×10⁸ to about 5.0×10⁸, about 5.0×10⁸ to about 7.5×10⁸, about 7.5×10⁸ to about 1.0×10⁹, and overlapping ranges thereof. In certain embodiments, the exosome dose is administered on a per kilogram basis, for example, about 1.0×10⁵ exosomes/kg to about 1.0×10⁹ exosomes/kg. In additional embodiments, exosomes are delivered in an amount based on the mass of the target tissue, for example about 1.0×10⁵ exosomes/gram of target tissue to about 1.0×10⁹ exosomes/gram of target tissue. In several embodiments, exosomes are administered based on a ratio of the number of exosomes the number of cells in a particular target tissue, for example exosome:target cell ratio ranging from about 10⁹:1 to about 1:1, including about 10⁸:1, about 10⁷:1, about 10⁶:1, about 10⁵:1, about 10⁴:1, about 10³:1, about 10²:1, about 10:1, and ratios in between these ratios. In additional embodiments, exosomes are administered in an amount about 10-fold to an amount of about 1,000,000-fold greater than the number of cells in the target tissue, including about 50-fold, about 100-fold, about 500-fold, about 1000-fold, about 10,000-fold, about 100,000-fold, about 500,000-fold, about 750,000-fold, and amounts in between these amounts. If the exosomes are to be administered in conjunction with the concurrent therapy (e.g., cells that can still shed exosomes, pharmaceutical therapy, nucleic acid therapy, and the like) the dose of exosomes administered can be adjusted accordingly (e.g., increased or decreased as needed to achieve the desired therapeutic effect).

In several embodiments, the exosomes are delivered in a single, bolus dose. In some embodiments, however, multiple doses of exosomes may be delivered. In certain embodiments, exosomes can be infused (or otherwise delivered) at a specified rate over time. In several embodiments, when exosomes are administered within a relatively short time frame after an adverse event (e.g., an injury or damaging event), their administration prevents the generation or progression of damage to a target tissue. For example, if exosomes are administered within about 20 to about 30 minutes, within about 30 to about 40 minutes, within about 40 to about 50 minutes, within about 50 to about 60 minutes post-adverse event, the damage or adverse impact on a tissue is reduced (as compared to tissues that were not treated at such early time points). In some embodiments, the administration is as soon as possible after an adverse event. In some embodiments the administration is as soon as practicable after an adverse event (e.g., once a subject has been stabilized in other respects). In several embodiments, administration is within about 1 to about 2 hours, within about 2 to about 3 hours, within about 3 to about 4 hours, within about 4 to about 5 hours, within about 5 to about 6 hours, within about 6 to about 8 hours, within about 8 to about 10 hours, within about 10 to about 12 hours, and overlapping ranges thereof. Administration at time points that occur longer after an adverse event are effective at preventing damage to tissue, in certain additional embodiments.

In some embodiments, the composition is in a parenteral dose form. In some embodiments, parenteral dosage forms is sterile or capable of being sterilized before administering to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration to a subject. Suitable excipients that can be used to provide parenteral dosage forms of CDC-derived exosomes include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

### Examples

It will be appreciated by persons skilled in the art that numerous variations and/or modifications may be made to the present invention as shown in the specific embodiments without departing from the spirit or scope of the present invention as broadly described. The present embodiments are, therefore, to be considered in all respects as illustrative and not restrictive. In order that the present invention may be readily understood and put into practical effect, particular preferred embodiments will now be described by way of the following non-limiting examples.

### A. Human CDC-EV Source

CDC-EVs from three different donors (Donor 1, Donor 2, and Donor 3) were obtained from Capricor Therapeutics, Inc. (Beverly Hills, CA). Samples Donor 1 and Donor 2 CDCs provided a limited supply of EVs that were utilized in subsequent in vitro coagulation studies. Due to consistent profiles between these two samples, and a need for larger EV yields for animal models, EVs derived from sample Donor 3 were utilized for the in vivo portion of this study.

### B. CDC Culture and EV Isolation

CDCs were prepared as described in US/2012/0315252, the disclosures of which are herein incorporated by reference in their entirety. In brief, heart biopsies were minced into small fragments and briefly digested with collagenase. Explants were then cultured on 20 mg/mL fibronectin-coated dishes. Stromal-like flat cells and phase-bright round cells grew out spontaneously from tissue fragments and reached confluency by 2-3 weeks. These cells were harvested using 0.25% trypsin and were cultured in suspension on 20 mg/mL poly-d-lysine to form self-aggregating cardiospheres. CDCs were obtained by plating and expanding the cardiospheres on a fibronectin-coated flask as an adherent monolayer culture. All cultures were maintained at 5% O₂, 5% CO₂ at 37° C., using IMDM basic medium supplemented with 10% FBS, 1% penicillin/streptomycin, and 0.1 mL 2-mercaptoethanol. CDCs were grown to 100% confluency on a fibronectin-coated flask to passage 5.

CDCs (passage 4) were thawed at 37° C. and cultured in T175 flasks using CDC media for three days at 37° C., 5% CO₂, 5% O₂. Cells were harvested on day three and 0.5 L PBS vessels were seeded with CDCs (6E6 cells/vessel) in 500 mL of serum free media (serum-free Iscove's Modified Dulbecco's Medium (IMDM) without phenol red medium; 21056023, ThermoFisher, Waltham, MA) with 5 g of CellBind microcarriers (Corning, Glendale, AZ). The vessels were incubated for three days at 37° C., 5% CO₂, and 5% O₂, with wheel rotation set at 20 RPM. On day three, the PBS vessels were moved to 37° C., 5% CO₂, and 20% O₂. On day six, Donor 3 vessels alone were incubated at 37° C., 5% CO₂, and 2% 02 (hypoxia condition) for 24 h. After 24 h incubation, 5 mL microcarrier-cell culture from each CDC container was collected for cell count and cell viability using trypan blue staining. The remaining culture medium from both flasks was filtered through 0.45 μm, 1 L bottle-top filters to remove microcarrier-cells and collected pre-filtered conditioned medium (pfCM). pfCM was concentrated using ultrafiltration by centrifugation (UFC; Donor 1 and Donor 2) or tangential flow filtration (TFF; Donor 3). For UFC, a 100 kDa centrifugation filter was applied (Amicon, Millipore Sigma, Burlington MA) according to manufacturer's protocol. For TFF isolation filtered conditioned medium was subjected to ultrafiltration using a 750-kDa cutoff TFF cartridge (MidiKros mPES 245 cm², D02-E750-10-S; Spectrum Labs, Rancho Dominguez, CA, USA). A feed flow rate of 100 mL/min was maintained throughout the filtration operation. The conditioned medium was concentrated and then buffer exchanged with PlasmaLyte-A. Exosomes in Plasmalyte-A were 0.2 μm filtered and stored at −80° C.

C. Exosome Derivation from 293F cells

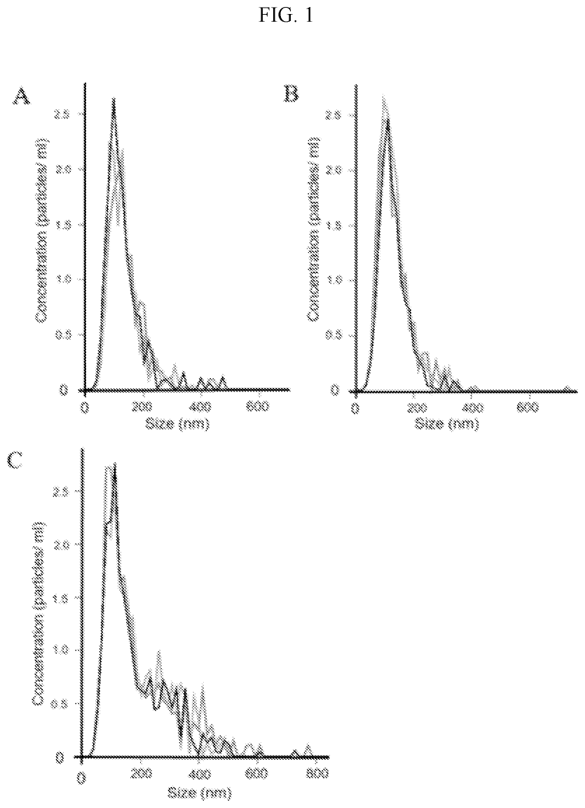

Exosomes were derived from suspension cultures of 293F cells at concentration of 1.5×10⁶ cells/mL (e.g., Thermo Fisher Cat #R79007) grown for 3 days in chemically defined 500 mLs media (e.g., FreeStyle™) in tissue culture shaker (110 rpm) flasks (FIG. 1). Following removal of cells and large cell debris by progressive centrifugation at 500×g (5 mins), the conditioned media was clarified by passage the total volume of 500 mLs through a 0.22 micron sterile filtration unit. Exosomes were recovered from this clarified tissue culture supernatant/conditioned media (CTCS/CM) by concentrating the volume using filtration, size exclusion chromatography, and a second round of concentrating filtration. The Exosomes were collected into total volume of 500 uls. The concentration and size was measured using NanoSight technology.

### D. Nanoparticle Tracking Analysis (NTA) of CDC-EVs

EVs were gently vortexed for 10 seconds and then bath sonicated for 10 minutes at 33° C. to ensure adequate vesicle dispersion in the solution prior to NTA analysis. NanoSight measurements were carried out in 0.02 μm filtered phosphate buffered saline solution (PBS) to remove any background particles and then visualized on an NS300 NanoSight (Malvern Panalytical, United Kingdom) instrument at ambient temperature. Camera settings were fixed for all measurements during the session (camera level of 15, camera gain of 366, and temperature of 21.7-22.2° C.). EVs were normalized by particle count for subsequent experiments.

Nanoparticle tracking analysis confirmed that the majority of isolated CDC-EVs fell between a size range of 30 to 400 nm (FIG. 1). The mean size distribution of CDC-EVs and their standard deviation are as follows: 132.5±2.7 nm for Donor 1 (FIG. 1A), 133.1±1.2 nm for Donor 2 (FIG. 1B), and 184.9±3.2 nm for Donor 3 (FIG. 1C). The respective modes for the isolated CDC-EVs are as follows: 96.8 nm (Donor 1), 106.6 nm (Donor 2), and 108.2 nm (Donor 3). Combined, the data demonstrates that, while the mean size distribution of CDC-EVs from Donor 3 are slightly larger than those from Donor 1 and Donor 2, the CDC-EVs are uniform in their mode across samples.

### E. Flow Cytometry

Flow cytometry (BD FACSCANTO II, BD Biosciences, San Jose, CA) to probe for procoagulant markers tissue factor (anti-CD142 (TF); PE, 12-1429-42, ThermoFisher) and phosphatidylserine (PS; Alexa Fluor-488, 16-256, Sigma-Aldrich) was performed as previously described.(33) 25 μg of total EV protein (roughly 1.3×10⁷ particles/mL of EVs) was used for the experiment. Samples were run as technical replicates.

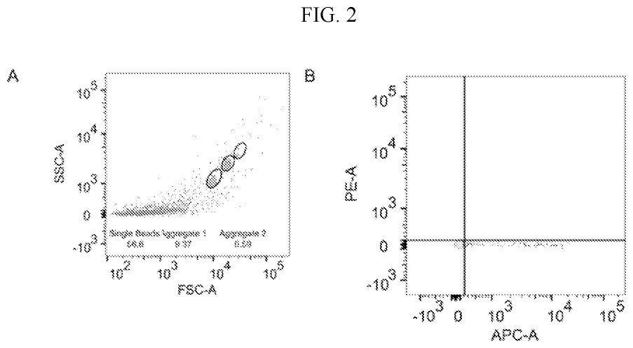

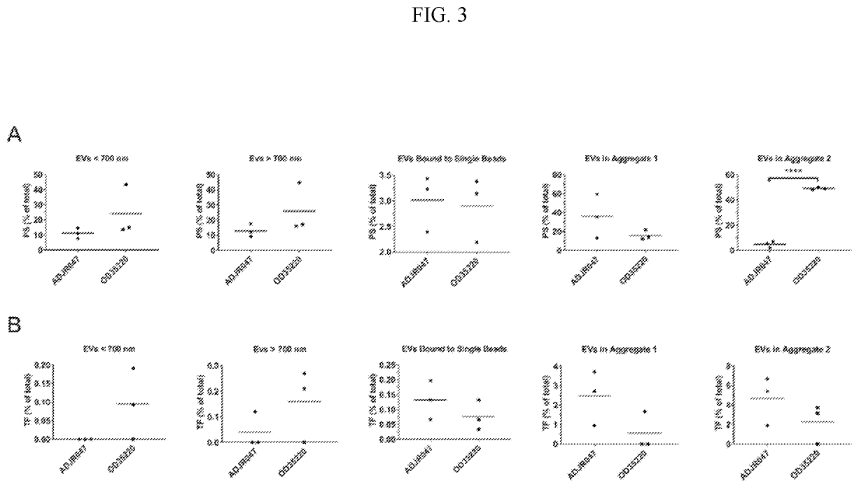

CDC-EVs analyzed by flow cytometry formed distinct groups for analysis as previously described in prior studies (FIG. 2).(33) Briefly, the flow groups formed were: (1) EV aggregates less than 700 nm (<700 nm), (2) EV aggregates between 700-1,000 nm (>700 nm), (3) EVs bound to single Dynabeads (single beads), (4) an aggregate of Dynabeads and EVs (aggregate 1), and (5) a larger aggregate of Dynabeads and EVs (aggregate 2). Overall, there were no significant trends in phosphatidylserine (PS) expression across flow groups (FIG. 3A). Within the “EVs in Aggregate 2” flow group, however, EVs from Donor 2 expressed significantly more PS than EVs from Donor 1. Similarly, there were no significant trends in tissue factor (TF) expression across and within flow groups (FIG. 3B).

### F. Pooled Platelet-Poor Plasma (PPP) Preparation

Platelet-poor plasma was prepared as previously described.(33) Blood components were collected by centrifugation of whole blood from healthy donors under an institute-approved standard operating procedure.

### G. Calibrated Thrombogram (CAT)

Thrombin activity was assayed as previously described using the calibrated thrombogram (Oude Maastricht, The Netherlands, Stago) with minor modifications.(33) All EV samples were normalized to 10 μg of total EV protein (roughly 5×10⁶ particles/mL). For EV samples, 60.04 μL of PPP was mixed with 19.96 μL of EVs suspended in serum free media (Plasmalyte-A; Baxter, Deerfield, IL, USA) in a 96-well plate. The samples were then mixed with 20 μL of MP reagent, containing phospholipids to support the triggering of thrombin generation by microparticles, but no TF (TS60.00, Thrombinoscope, BM Maastricht, the Netherlands). 4-(2-hydroxyethyl)-1-piperazineethanesulfonic acid (HEPES) buffer and PPP reagent (containing a mixture of TF and phospholipids; TS30.00, Thrombinoscope) were used as negative and positive controls, as previously described. Samples were run as technical replicates.

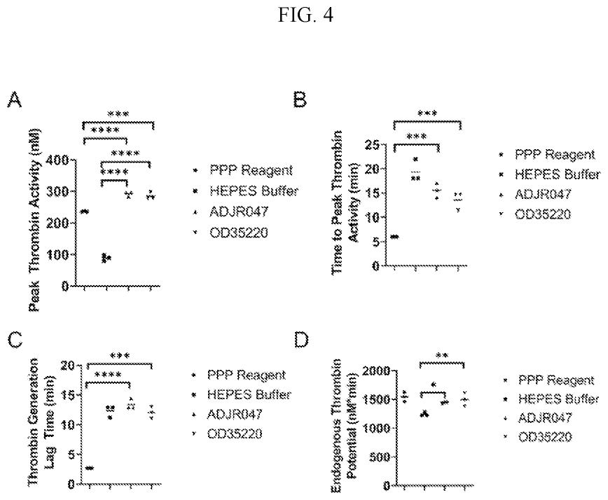

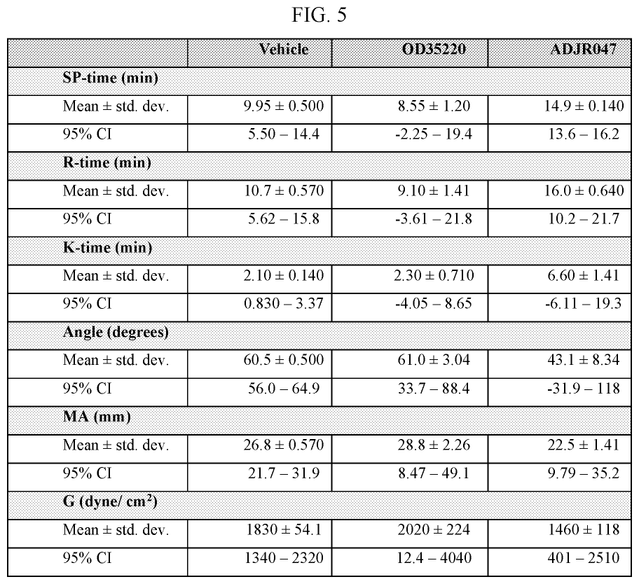

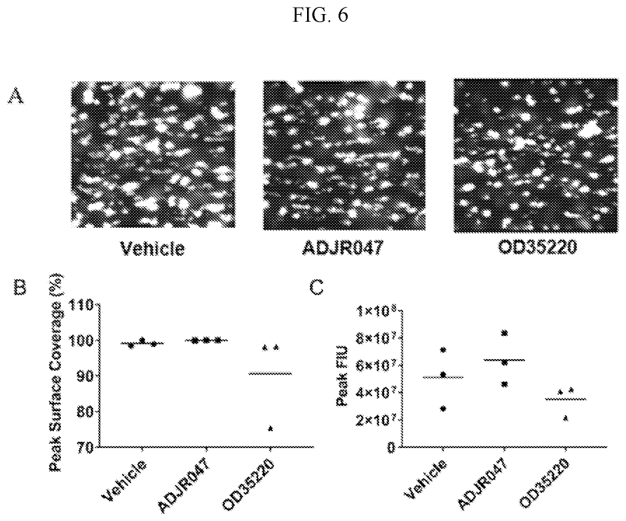

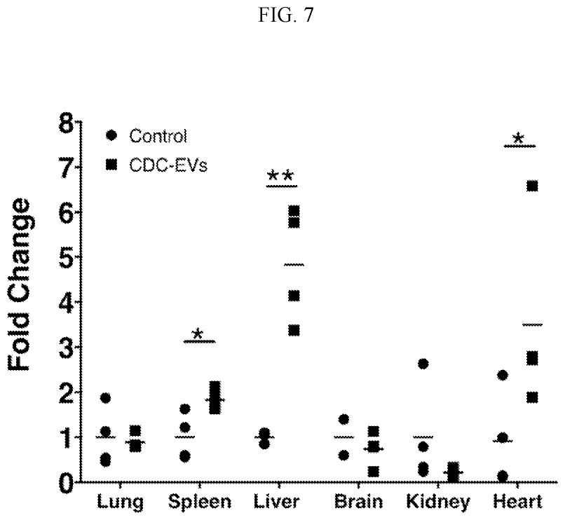

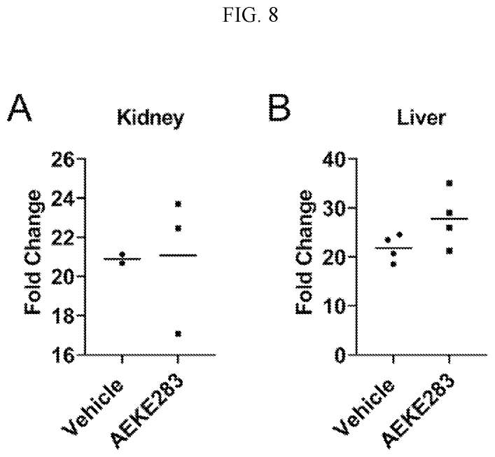

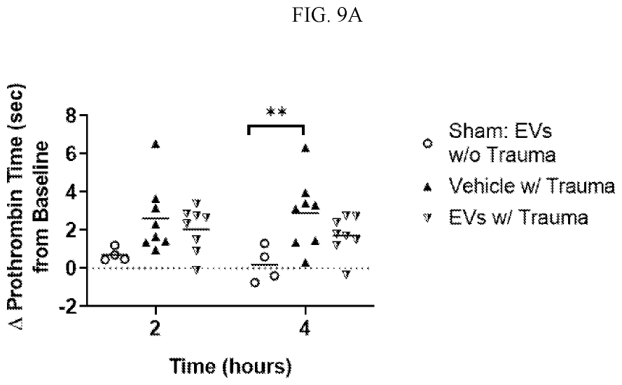

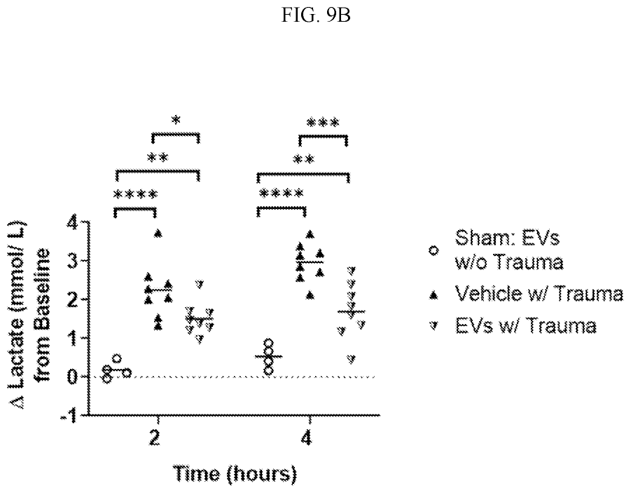

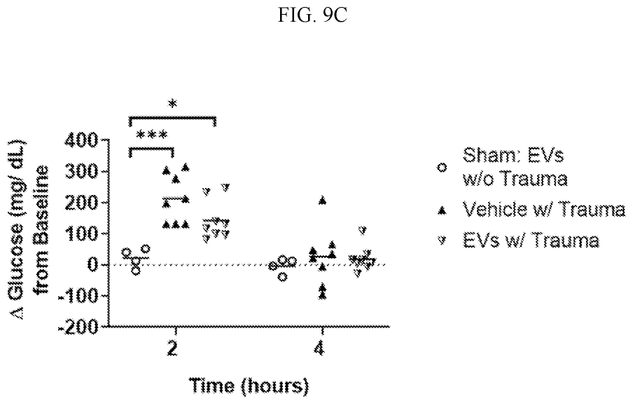

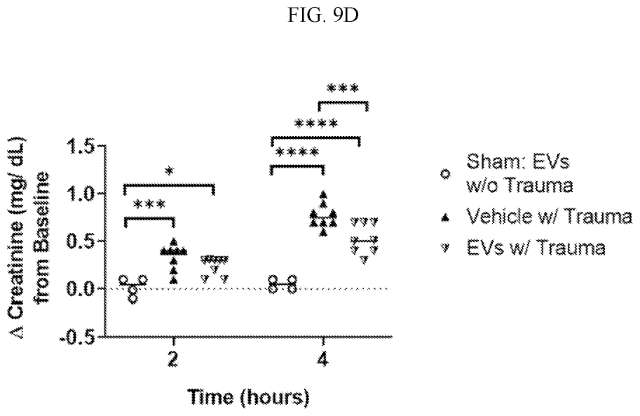

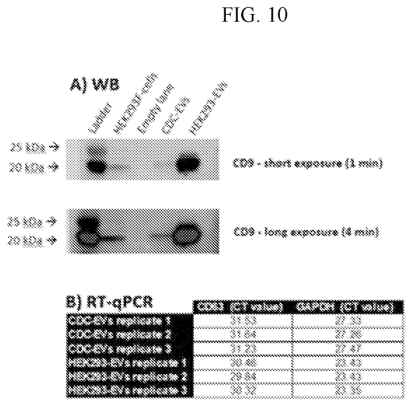

Whether or not CDC-EVs had the potential to generate thrombin activity was evaluated using CAT (FIG. 4). Analysis by one-way ANOVA indicated that both EV groups significantly increased peak thrombin activity (FIG. 4A) compared to the negative control of HEPES buffer (p<0.0001 for both) and the positive control of PPP reagent (p<0.0001 for Donor 1 and p<0.001 for Donor 2). Additionally, the two CDC-EV groups generated significantly more total thrombin (endogenous thrombin potential or ETP; FIG. 4D) than the negative control (p<0.05 for Donor 1 and p<0.01 for Donor 2), but not for the positive control (p>0.05). Conversely, CDC-EVs took longer to generate thrombin activity, significantly increasing time to peak thrombin activity (FIG. 4B; p<0.001 for both) and thrombin generation lag time (FIG. 4C; p<0.0001 for Donor 1 and p<0.001 for Donor 2) compared to PPP reagent.

CDC-EV dose did not impact clot formation in a meaningful manner, as measured by thromboelastogram (TEG) (FIG. 5). CDC-EVs isolated from strain Donor 2 displayed similar clotting parameters compared to the vehicle control, while CDC-EVs isolated from Donor 1 formed slower and weaker clots compared to the control.

### H. Flow-Based Adhesion (Bioflux)

Flow-based adhesion to determine the effects of EVs on platelet-collagen interaction was performed as previously described using the Bioflux (Fluxion Biosciences, San Francisco, CA).(33) 100 μg of total EV protein (roughly 5×10⁷ particles/mL) was used for the experiment. Briefly, channels were coated with 100 mg/mL type I collagen from equine tendon (Helena Laboratories, Beaumont, TX), rinsed with PBS, and then blocked with 0.5% bovine serum albumin (BSA). Platelets were stained with MitoTracker Red CMXRos TRITC (ThermoFisher) before re-suspension in plasma at a concentration of 200,000/mL and 40% hematocrit. Samples were perfused through collagen-coated wells at a wall shear rate of 720/s. Samples were run as donor replicates.

The effect of CDC-EVs on platelet adhesion to a collagen coated surface under physiologically relevant flow conditions were evaluated (FIG. 6A). Platelets in reconstituted blood were perfused at an arterial shear rate meant to mimic changes in shear rate associated with trauma through channels coated with collagen. Data analysis by one-way ANOVA indicated that there were no significant effects (p<0.05) of CDC-EVs from both Donor 1 and Donor 2 samples on peak platelet surface coverage (representing platelet adhesion; FIG. 6B) and peak platelet fluorescent intensity units FIU (representing platelet aggregation; FIG. 6C).

### I. Rat Polytrauma and Hemorrhage Model

Research was conducted in compliance with the Animal Welfare Act, the implementing Animal Welfare regulations, and the principles of the Guide for the Care and Use of Laboratory Animals, National Research Council. The facility's Institutional Animal Care and Use Committee approved all research conducted in this study. The facility where this research was conducted is fully accredited by the AAALAC. Sprague-Dawley rats (350-450 g) were anesthetized with 1-2% isoflurane/100% oxygen through a nose cone, and allowed to breathe spontaneously. Cannulas were placed in the left femoral artery and vein for monitoring arterial blood pressure and for withdrawing blood and injection. Polytrauma was performed as previously described.(37) Briefly, a midline incision was made through the abdominal skin and underlying muscle layers. The right and medial lobes of the liver received three crush injuries each using a clamp covered with Silastic tubing. A 10 cm section of small intestines anterior to the cecum was isolated and run gently through the same clamp. The intestines and liver were replaced, and the abdominal incision closed in two layers with sutures. Femur fracture was accomplished by dropping six stainless-steel balls (65 g each) stacked together, from 36 inches (91.44 cm) through a guide tube (1 inch, or 2.54 cm, internal diameter) to impact on a rounded aluminum blade resting on the mid-right femur of the right leg that was suspended on two aluminum stands, one under the hip and one under the knee. A large hemostat (5 inch, or 12.7 cm, tongs) was used to clamp the muscle of the right leg 10 times. The rats were then bled in a controlled manner through the venous cannula to a mean arterial pressure of 40 mmHg which was maintained until 40% of estimated blood volume was removed. Hemorrhage was completed within 30 min of trauma, then blood pressure and heart rate were allowed to freely compensate. No resuscitation fluid was given. Blood volume was estimated as 6% of body weight±0.77 mL. All rats survived the 240 min experimental period, which was immediately followed by euthanasia. Rats treated with EVs for an equivalent period of time but without trauma were used as a sham control in order to isolate the specific effects of the treatment, as opposed to incidental effects caused by operative procedures such as anesthesia and incisional trauma.

### J. EV Injection and Sample Collection

EVs at a concentration of 1×10¹⁰ particles/mL (suspended in 1 mL of Plasmalyte-A), or an equal volume of Plasmalyte-A, were injected into the femoral vein one hour post trauma. Whole blood samples (in 20 mM sodium citrate) were taken before trauma (after cannulation; baseline) and at 60 and 180 min after injection of EVs from the femoral vein.

### K. Biodistribution Analysis

Nine weeks old C57BL10 female mice (Jackson Lab) were used in this study. For intravenous (IV) injection (jugular vein), four mice were infused with plasmalyte (100 μL) and four mice with human CDC-EVs (1e10 particles per mouse) through the jugular vein. All mice were sacrificed one-hour post IV injections, and different organs were collected and preserved in RNAlater (ThermoFisher) at −20° C. until further processing was performed.

Organs were homogenized in Qiazol (Qiagen) using Bead Ruptor 12 (OMNI International) with RNase free steel beads. RNA was isolated using miRNeasy kit (Qiagen), and QuantiMir Kit (Systems Biosciences) was used for reverse transcription. qPCR was done with SYBR green on QuantStudio 12K Flex instrument (Applied Biosystems) with the QuantiMir universal reverse primer, hY4f forward primer (5′-GGTCCGATGGTAGTGGGTTATCAG-3′), and mouse U6 forward primer (5′-TGGCCCCTGCGCAAGGATG-3′) for the housekeeping gene. Fold change was calculated using 2(−ΔΔCt) by comparing the ΔCt of each tissue to the ΔCt of that tissue from the Plasmalyte-injected mice.

EV biodistribution was first assessed in rats (FIG. 8) and mice (FIG. 7) as a tool to help determine their organ protective benefits. CDC-EVs were undetectable in rats following trauma, but were detected in mice primarily in the liver and heart one hour after administration with 4.8 and 3.8 fold increase respectively when compared with control mice (p<0.01 and p<0.05). A moderate accumulation of CDC-EVs was observed in the spleen of infused mice with 1.8 fold increase when compared with control samples (p<0.05) (FIG. 7). There were no significant differences in CDC-EV accumulation in the lung, brain, and kidney compared to control.

### L. Prothrombin Time (PT)

Prothrombin times (PT) were assessed on a Stago ST4 Coagulation Analyzer (Stago, Parsippany, NJ) using 50 μL whole blood, following manufacturer protocol.

At two hours after trauma, neither rats treated with vehicle nor CDC-EVs significantly increased prothrombin times compared to sham controls (FIG. 9A; p>0.05). However, at four hours, rats treated with vehicle maintained significantly higher prothrombin times compared to sham rats (p<0.01). CDC-EV treated rats, on the other hand, showed no significant changes in prothrombin time compared to sham rats (p>0.05).

M. Lactate, Glucose, and Creatinine Measurements Using i-STAT

Whole blood was used for i-STAT analysis (i-STAT Handheld, Abbot Laboratories, Chicago, IL) utilizing the CG4+ (03P85-50) and Chem8+ (09P31-25) test cartridges.

At two hours post trauma, both rats treated with vehicle and CDC-EVs significantly increased lactate levels compared to sham rats (FIG. 9B; p<0.0001 and p<0.01, respectively). Additionally, rats at two hours showed significantly lower lactate levels when treated with CDC-EVs compared to vehicle (p<0.05). Similar to the two hour time-point, rats treated with vehicle and CDC-EVs significantly increased lactate levels compared to sham rats (p<0.0001 and p<respectively). However, CDC-EVs significantly decreased lactate levels compared to vehicle (p<0.001), evidenced by the decreasing average between the time-points.

At two hours post trauma, rats treated with vehicle and CDC-EVs both significantly increased glucose levels compared to sham rats (FIG. 9C; p<0.001 and p<0.05, respectively). Rats treated with CDC-EVs increased glucose levels less so than those treated with vehicle, compared to sham at the two hour time-point. At the four hour time-point, however, the averages of all values return to baseline values.

Creatinine levels at two hours were significantly elevated, following treatment with vehicle and CDC-EVs, compared to sham rats (FIG. 9D; p<0.001 and p<0.05, respectively). Rats treated with vehicle increased creatinine levels more so than CDC-EVs, at the two hour time-point compared to sham. At four hours, average values of the experimental groups continued to rise (p<0.0001 for both vehicle and CDC-EV groups). Between treatment groups, however, it was observed that CDC-EVs significantly lowered creatinine levels compared to vehicle (p<0.001).

### N. Statistical Analysis

NanoSight data sets are depicted as line graphs. All other data sets are depicted with

individual data points alongside the mean. Prothrombin time, and the levels of lactate, glucose level, and creatinine at 60, 180 min after EVs injection were presented as the difference to the respective levels at baseline. Statistical analyses were performed using GraphPad Prism (version 7, GraphPad Software, La Jolla, CA). A resulting p-value of less than or equal to 0.05 was considered to be significant. NanoSight data sets (n=3 technical replicates) were analyzed using basic parameters (mean and standard deviation). Flow cytometry (n=3 technical replicates) data sets were analyzed using a two-tailed unpaired t test. CAT (n=3 technical replicates using pooled platelet poor plasma) and bioflux data sets (n=3 donor replicates in duplicate) were analyzed using a one-way ANOVA followed by a Tukey correction for multiple comparisons. Prothrombin times as well as lactate, glucose, and creatinine concentration data sets (n=4, 8, and 8, for rats treated as a sham, with EVs, and with vehicle, respectively) were analyzed using a repeated measures two-way ANOVA with a Tukey correction for multiple comparisons.

qPCR analysis for biodistribution analysis was performed in triplicates using 4 mice in each group. Data sets were analyzed using a one-tailed, two-sample unequal variance t test.

### O. Results

Herein, the present inventors observed that while CDC-EVs are functionally thrombogenic, they are much slower to generate thrombin than the positive control (FIGS. 4B and 4C) and do not generate amounts compared to the positive control that is likely to be clinically significant (FIG. 4D). The delayed kinetics in thrombin generation of CDC-EVs was also evidenced by their tendency to form clots at a rate similar to the vehicle negative control (FIG. 5). Following suit, CDC-EVs did not affect platelet adhesion and aggregation in a flow-based in vitro assay simulating clotting over an exposed layer of collagen at damaged sub-endothelium (FIG. 6). The low thrombogenic potential of CDC-EVs is further explained by their relatively low expression of phosphatidylserine (FIG. 3A) and tissue factor (FIG. 3B) when compared to MSC-EVs in vitro.(33) These combined aspects potentially make CDC-EVs a viable and safe candidate for future application in the treatment of trauma with coagulopathy.

Administration of CDC-EVs did not significantly elongate prothrombin times in sham rats, and did not enhance coagulopathy in rats with acute traumatic coagulopathy induced by polytrauma and hemorrhagic shock (FIG. 9A). Surprisingly, the present inventors found that CDC-EVs not only reduced the elevation of lactate, but also other key parameters associated with trauma and shock severity: glucose (while not significant, there was a trend of lower levels compared to vehicle) and creatinine (FIGS. 9B-9D). Serum lactate level is a reliable predictor of patient mortality with early clearance correlating with decreased levels of shock severity.(5, 11-17) The kidney and liver are two major organs that regulate lactate levels, more specifically, lactate metabolism and consumption, respectively.(39) Together, the kidney and liver can account for approximately 35-42% of lactate removal from the body.(39) As a result of these findings, the present inventors sought to determine whether or not the CDC-EVs were distributed to the liver and kidneys of the rats, where they might either promote or modulate lactate consumption through LDH activity, or preserve organ function following injury. However, the present inventors were unable to detect increased CDC-EV biodistribution in rat liver and kidneys following infusion through the femoral vein, as RNA specific to CDC-EVs was difficult to detect through a real-time polymerase chain reaction (RT-PCR-based) method (FIG. 2). The present inventors were, however, able to determine the biodistribution of CDC-EVs in a mouse model after jugular vein administration using RT-PCR (FIG. 7). Data demonstrated that after 1 hour post-injection, there was a significant amount of CDC-EVs sequestered in the liver compared to control, as well as an un-significant portion (compared to control) of CDC-EVs sequestered in the kidneys. From this, it can be speculated that CDC-EVs tend to primarily sequester to the liver following intravenous administration in rat models, with a minor portion sequestering to the kidneys.

This is the first study to characterize the procoagulant activity of EVs derived from cardiosphere-derived cells (CDC-EVs), and to test their safety and potential in a rat model of acute traumatic coagulopathy induced by polytrauma and hemorrhagic shock. These experimental results are of great relevance to the development of EV products for use in combat casualty care, as these studies show that CDC-EVs have the potential to be an anti-shock therapeutic if administered early, ideally within 0-2 hours of injury.(12) Compared to CDCs, CDC-EVs offer therapeutic benefits that are important in the development of alternative blood products to treat trauma and hemorrhagic shock. These results demonstrate that CDC-EVs likely have therapeutic utility in trauma and hemorrhagic shock beyond their reported cardio-protective benefits.

### REFERENCES

1. Payal P, Sonu G, Anil K G, et al. Management of polytrauma patients in emergency department: An experience of a tertiary care health institution of northern India. World J Emerg Med. 2013; 4(1):15-9.
2. Wu X, Darlington D N, Cap A P. Procoagulant and fibrinolytic activity after polytrauma in rat. Am J Physiol Regul Integr Comp Physiol. 2016; 310(4):R323-9.
3. Choi J Y, Lee W H, Yoo T K, et al. A new severity predicting index for hemorrhagic shock using lactate concentration and peripheral perfusion in a rat model. Shock. 2012; 38(6):635-41.
4. Mevius H, van Dijk M, Numanoglu A, et al. Article Commentary: The Management of Pediatric Polytrauma: Review. Clinical Medicine Insights: Trauma and Intensive Medicine. 2014; 5:27-37.
5. Wah K C, Man C W, Yuen Ha W J, et al. Evolving Frontiers in Severe Polytrauma Management—Refining the Essential Principles. Malays J Med Sci. 2012; 20(1):1-12.
6. McCullough A L, Haycock J C, Forward D P, et al. Early management of the severely injured major trauma patient. Br J Anaesth. 2014; 113(2):234-41.
7. Eakins J. Blood Glucose Control in the Trauma Patient. JDST. 2009; 3(6):1373-6.
8 Heffernan D S, Monaghan S F, Thakkar R K, et al. Failure to normalize lymphopenia following trauma is associated with increased mortality, independent of the leukocytosis pattern. Crit Care. 2012; 16(1):1-10.
9. Cortes-Samaca C A, Melendez-Flórez H J, Robles S A, et al. Base deficit, lactate clearance, and shock index as predictors of morbidity and mortality in multiple-trauma patients. Colombian J Anesth. 2018; 46(3):208-15.
10. Hemanthakumar, Velmurugan K, Sathish M. Serum Lactate—An indicator of morbidity & mortality in Polytrauma patients. IOSR-JDMS. 2017; 16(10):17-25.
11. Lamichhane P, Shrestha S, Banskota B, et al. Serum Lactate—An indicator of morbidity and mortality in polytrauma and multi-trauma patients. NOAJ. 2011; 2(1):7-13.
12. Régnier M-A, Raux M, Le Manach Y, et al. Prognostic Significance of Blood Lactate and Lactate Clearance in Trauma Patients. Anesthesiology. 2012; 117(6):1276-88.
13. Laverde Sabogal C E, Correa Rivera A F, Joya Higuera A Y. Lactate and base deficit in trauma: Prognostic value. Colombian J Anesth. 2014; 42(1):60-4.
14. Soller B, Zou F, Prince M D, et al. Comparison of Noninvasive pH and Blood Lactate as Predictors of Mortality in a Swine Hemorrhagic Shock with Restricted Volume Resuscitation Model. Shock. 2015; 44(1):90-5.
15. Lee S M, An W S. New clinical criteria for septic shock: serum lactate level as new emerging vital sign. J Thorac Dis. 2016; 8(7):1388-90.
16. Ellingson K D, Sapiano M R P, Haass K A, et al. Continued decline in blood collection and transfusion in the United States-2015. Transfusion. 2017; 57(2):1588-98.
17. Roberts N, James S, Delaney M, et al. The global need and availability of blood products: a modelling study. The Lancet Haematology. 2019:1-10.
18. Sarko D K, McKinney C E. Exosomes: Origins and Therapeutic Potential for Neurodegenerative Disease. Front Neurosci. 2017; 11(82):1-7.
19. Phinney D G, Pittenger M F. Concise Review: MSC-Derived Exosomes for Cell-Free Therapy. Stem Cells. 2017; 35(4):851-8.
20. Nakamura Y, Miyaki S, Ishitobi H, et al. Mesenchymal-stem-cell-derived exosomes accelerate skeletal muscle regeneration. FEBS Lett. 2015; 589(11):1257-65.
21. Petho A, Chen Y, George A. Exosomes in Extracellular Matrix Bone Biology. Curr Osteoporos Rep. 2018; 16(1):58-64.
22. Ko J, Carpenter E, Issadore D. Detection and isolation of circulating exosomes and microvesicles for cancer monitoring and diagnostics using micro-/nano-based devices. Analyst. 2016; 141(2):450-60.
23. Katsuda T, Tsuchiya R, Kosaka N, et al. Human adipose tissue-derived mesenchymal stem cells secrete functional neprilysin-bound exosomes. Sci Rep. 2013; 3:1-11.22.
24. Kowal J, Arras G, Colombo M, et al. Proteomic comparison defines novel markers to characterize heterogeneous populations of extracellular vesicle subtypes. J Cell Bio. 2016:E968-977.
25. Zaborowski M P, Balaj L, Breakefield X O, et al. Extracellular Vesicles: Composition, Biological Relevance, and Methods of Study. Bioscience. 2015; 65(8):783-97.
26. Haider H, Ashraf M. Strategies to promote donor cell survival: combining preconditioning approach with stem cell transplantation. J Mol Cell Cardiol. 2008; 45(4):554-66.
27. Prockop D J. Repair of tissues by adult stem/progenitor cells (MSCs): controversies, myths, and changing paradigms. Mol Ther. 2009; 17(6):939-46.
28. Barile L, Lionetti V, Cervio E, et al. Extracellular vesicles from human cardiac progenitor cells inhibit cardiomyocyte apoptosis and improve cardiac function after myocardial infarction. Cardiovasc Res. 2014; 103(4):530-41.
29. Chen L, Wang Y, Pan Y, et al. Cardiac progenitor-derived exosomes protect ischemic myocardium from acute ischemia/reperfusion injury. Biochem Biophys Res Commun. 2013; 431(3): 566-71.
30. Deddens J C, Vrijsen K R, Girao H, et al. Cardiac-released extracellular vesicles can activate endothelial cells. Ann Transl Med. 2017; 5(3):1-3.
31. Sahoo S, Losordo D W. Exosomes and cardiac repair after myocardial infarction. Circ Res. 2014; 114(2): 333-44.
32. Garcia N A, Moncayo-Arlandi J, Sepulveda P, et al. Cardiomyocyte exosomes regulate glycolytic flux in endothelium by direct transfer of GLUT transporters and glycolytic enzymes. Cardiovasc Res. 2016; 109(3):397-408.
33. Chance T C, Rathbone C R, Kamucheka R M, et al. The effects of cell type and culture condition on the procoagulant activity of human mesenchymal stromal cell-derived extracellular vesicles. J Trauma Acute Care Surg. 2019; 87(1S Suppl 1):574-582.
34. Silachev D N, Goryunov K V, Shpilyuk M A, et al. Effect of MSCs and MSC-Derived Extracellular Vesicles on Human Blood Coagulation. Cells. 2019; 8(3):258-81.
35. Puranik G N, Verma T Y P, Pandit G A. The Study of Coagulation Parameters in Polytrauma Patients and Their Effects on Outcome. J Hematol. 2018; 7(3):107-11.
36. Mica L, Albrecht K. Independent Predictors of Early Death of Polytrauma Patients: An Analysis of 696 Patients. J Trauma Treat. 2012; 01(03):1-5.
37. Darlington D N, Craig T, Gonzales M D, et al. Acute coagulopathy of trauma in the rat. Shock. 2013:39:440-46.
38. Luther T and Mackman N. Tissue Factor in the Heart Multiple Roles in Hemostasis, Thrombosis, and Inflammation. TCM. 2001; 11(08):307-12.
39. Bellomo, R. Bench-to-bedside review: Lactate and the kidney. Crit. Care. 2002; 6(4):1-5.

1. A method of treating polytrauma in a mammalian subject in need thereof, the method comprising the step of administering to the subject a therapeutically effective amount of extracellular vesicles derived from cardiosphere-derived cells (CDC-EVs) immediately or within a short time period after polytrauma, wherein said polytrauma is associated with coagulopathy and hemorrhagic shock.

2. The method according to claim 1, wherein parameters selected from lactate, glucose and creatinine, and combinations thereof that are monitored in the subject, are observed to decrease after treating the subject with the therapeutically effective amount of CDC-EVs.

3. A method of decreasing the risks associated with polytrauma in a mammalian subject suffering from polytrauma comprising the step of: administering to the subject a therapeutically effective amount of extracellular vesicles derived from cardiosphere-derived cells (CDC-EVs) immediately or within a short time period after polytrauma, wherein said polytrauma is associated with coagulopathy and hemorrhagic shock.

4. A method of treating trauma in a subject in need thereof, the method comprising the step of administering to the subject a therapeutically effective amount of extracellular vesicles derived from cardiosphere-derived cells (CDC-EVs).

5. The method according to claim 5, wherein said trauma is associated with, or is coupled with, or is induced by, coagulopathy and/or hemorrhagic shock.

6. A method of treating coagulopathy in a subject in need thereof, the method comprising the step of administering to the subject a therapeutically effective amount of extracellular vesicles derived from cardiosphere-derived cells (CDC-EVs).

7. The method according to claim 6, wherein said coagulopathy is acute coagulopathy.

8. The method according to claim 6 or claim 7, wherein said coagulopathy is associated with, or is coupled with, or is induced by, trauma and/or hemorrhagic shock.

9. A method of decreasing lactate, glucose and/or creatinine levels in a subject suffering from trauma, the method comprising administering to the subject a therapeutically effective amount of extracellular vesicles derived from cardiosphere-derived cells (CDC-EVs).

10. The method according to claim 9, wherein said trauma is associated with, or is coupled with, or is induced by, coagulopathy and/or hemorrhagic shock.

11. A method of decreasing lactate, glucose and/or creatinine levels in a subject suffering from coagulopathy, the method comprising administering to the subject a therapeutically effective amount of extracellular vesicles derived from cardiosphere-derived cells (CDC-EVs).

12. The method according to claim 11, wherein said coagulopathy is acute coagulopathy.

13. The method according to claim 11 or claim 12, wherein said coagulopathy is associated with, or is coupled with, or is induced by, trauma and/or hemorrhagic shock.

14. A method of decreasing the risk of shock in a subject suffering from trauma, the method comprising administering to the subject a therapeutically effective amount of extracellular vesicles derived from cardiosphere-derived cells (CDC-EVs).

15. The method according to claim 14, wherein said trauma is associated with, or is coupled with, or is induced by, coagulopathy and/or hemorrhagic shock.

16. A method of decreasing the risk of shock in a subject suffering from coagulopathy, the method comprising administering to the subject a therapeutically effective amount of extracellular vesicles derived from cardiosphere-derived cells (CDC-EVs).

17. The method according to claim 16, wherein said coagulopathy is acute coagulopathy.

18. The method according to claim 16 or claim 17, wherein said coagulopathy is associated with, or is coupled with, or is induced by, trauma and/or hemorrhagic shock.

19. A method of treating trauma to an organ in a subject in need thereof, the method comprising administering to the subject a therapeutically effective amount of extracellular vesicles derived from cardiosphere-derived cells (CDC-EVs).

20. The method according to claim 19, wherein said organ is selected from the liver, kidneys, intestines, heart, arteries, veins, stomach, lungs, brain, spinal cord, cerebellum, nerves, placenta, uterus, spleen, bladder, esophagus, pancreas, colon, rectum, sex organs, pharynx, larynx, trachea, bronchi, salivary glands, diaphragm, skeletal muscles, bones, bone marrow, cartilage, ligaments, tendons, lymphatic vessels, ureters, urethra, endocrine glands, blood vessels, and gallbladder.

21. The method according to any of claims 4, 5, 8-10, 13-15 and 18-20, wherein said trauma is polytrauma.

22. The method according to claim 21, wherein said trauma is acute trauma.

23. The method according to claim 22, wherein said acute trauma is acute polytrauma.

24. The method according to any of claims 1-23, wherein the lactate, glucose and/or creatinine levels in the subject are decreased upon being treated.

25. The method according to any of claims 1-24, wherein said effective amount of CDC-EVs is administered to the subject immediately after trauma and/or hemorrhagic shock.

26. The method according to claim 25, wherein said effective amount of CDC-EVs is administered to the subject within 1 to 2 hours after trauma and/or hemorrhagic shock.

27. The method according to claim 25, wherein said effective amount of CDC-EVs is administered to the subject within 12 hours after trauma and/or hemorrhagic shock.

28. The method according to claim 25, wherein said effective amount of CDC-EVs is administered to the subject within 24 hours after trauma and/or hemorrhagic shock.

29. The method according to any of claims 1-28, wherein said therapeutically effective amount of CDC-EVs is administered to the subject systemically or locally.

30. The method according to any of claims 1-29, wherein said extracellular vesicles are exosomes, microvesicles, membrane particles, membrane vesicles, exosome-like vesicles, ectosomes, ectosome-like vesicles, or exovesicles.

31. The method according to any of claims 1-30, wherein said subject is a mammalian subject.

32. The method according to claim 31, wherein said mammalian subject is a human subject.

33. The method according to any of claims 4, 5, 8-10, 13-15 and 18-32, wherein said trauma comprises one or more organ system failure.

34. The method according to claim 33, wherein said one or more organ system failure comprises liver and/or kidney failure.

35. A formulation comprising extracellular vesicles derived from cardiosphere-derived cells (CDC-EVs) and at least one pharmaceutically acceptable carrier for use in treating polytrauma in a subject in need thereof, wherein said polytrauma is associated with coagulopathy and hemorrhagic shock.

36. A formulation comprising extracellular vesicles derived from cardiosphere-derived cells (CDC-EVs) for use in treating trauma in a subject in need thereof.

37. The formulation according to claim 36, wherein said trauma is associated with, or is coupled with, or is induced by, coagulopathy and/or hemorrhagic shock.

38. A formulation comprising extracellular vesicles derived from cardiosphere-derived cells (CDC-EVs) for use in treating coagulopathy in a subject in need thereof.

39. The formulation according to claim 38, wherein said coagulopathy is acute coagulopathy.

40. The formulation according to claim 38 or claim 39, wherein said coagulopathy is associated with, or is coupled with, or is induced by, trauma and/or hemorrhagic shock.

41. A formulation comprising extracellular vesicles derived from cardiosphere-derived cells (CDC-EVs) for use in a method of decreasing the lactate, glucose and/or creatinine levels in a subject suffering from trauma, the method comprising administering to the subject a therapeutically effective amount of CDC-EVs.

42. The formulation according to claim 41, wherein said trauma is associated with, or is coupled with, or is induced by, coagulopathy and/or hemorrhagic shock.

43. A formulation comprising extracellular vesicles derived from cardiosphere-derived cells (CDC-EVs) for use in a method of in decreasing the lactate, glucose and/or creatinine levels in a subject suffering from coagulopathy, the method comprising administering to the subject a therapeutically effective amount of CDC-EVs.

44. The formulation according to claim 43, wherein said coagulopathy is acute coagulopathy.

45. The formulation according to claim 43 or claim 44, wherein said coagulopathy is associated with, or is coupled with, or is induced by, trauma and/or hemorrhagic shock.

46. A formulation comprising extracellular vesicles derived from cardiosphere-derived cells (CDC-EVs) for use in a method of in decreasing the risk of shock in a subject suffering from trauma, the method comprising administering to the subject a therapeutically effective amount of CDC-EVs.

47. The formulation according to claim 46, wherein said trauma is associated with, or is coupled with, or is induced by, coagulopathy and/or hemorrhagic shock.

48. A formulation comprising extracellular vesicles derived from cardiosphere-derived cells (CDC-EVs) for use in a method of decreasing the risk of shock in a subject suffering from coagulopathy, the method comprising administering to the subject a therapeutically effective amount of CDC-EVs.

49. The formulation according to claim 48, wherein said coagulopathy is acute coagulopathy.

50. The formulation according to claim 48 or claim 49, wherein said coagulopathy is associated with, or is coupled with, or is induced by, trauma and/or hemorrhagic shock.

51. A formulation comprising extracellular vesicles derived from cardiosphere-derived cells (CDC-EVs) for use in a method of treating trauma to an organ in a subject in need thereof, the method comprising administering to the subject a therapeutically effective amount of CDC-EVs.

52. The formulation according to claim 51, wherein said organ is selected from the liver, kidneys, intestines, heart, arteries, veins, stomach, lungs, brain, spinal cord, cerebellum, nerves, placenta, uterus, spleen, bladder, esophagus, pancreas, colon, rectum, sex organs, pharynx, larynx, trachea, bronchi, salivary glands, diaphragm, skeletal muscles, bones, bone marrow, cartilage, ligaments, tendons, lymphatic vessels, ureters, urethra, endocrine glands, blood vessels, and gallbladder.

53. The formulation according to claim 51 or claim 52, wherein said organ is the liver and/or the kidneys.

54. The formulation according to any of claims 36, 37, 40-42, 45-47 and 50-53, wherein said trauma is polytrauma.

55. The formulation according to claim 54, wherein said trauma is acute trauma.

56. The formulation according to claim 55, wherein said acute trauma is acute polytrauma.

57. The formulation according to any of claims 35-56, wherein the lactate, glucose and/or creatinine levels in the subject are decreased upon being treated with a therapeutically effective amount of CDC-EVs.

58. The formulation according to any of claims 35-57, wherein said effective amount of CDC-EVs is administered to the subject immediately after trauma and/or hemorrhagic shock.

59. The formulation according to claim 58, wherein said effective amount of CDC-EVs is administered to the subject within 1 to 2 hours after trauma and/or hemorrhagic shock.

60. The formulation according to claim 58, wherein said effective amount of CDC-EVs is administered to the subject within 12 hours after trauma and/or hemorrhagic shock.

61. The formulation according to claim 58, wherein said effective amount of CDC-EVs is administered to the subject within 24 hours after trauma and/or hemorrhagic shock.

62. The formulation according to any of claims 41-61, wherein said therapeutically effective amount of CDC-EVs is administered to the subject systemically or locally.

63. The formulation according to any of claims 35-62, wherein said extracellular vesicles are exosomes, microvesicles, membrane particles, membrane vesicles, exosome-like vesicles, ectosomes, ectosome-like vesicles, or exovesicles.

64. The formulation according to any of claims 35-63, wherein said subject is a mammalian subject.

65. The formulation according to claim 64, wherein said mammalian subject is a human subject.

66. The formulation according to any of claims 35-65, wherein said formulation is suitable for use in combat casualty care.

67. The formulation according to any of claims 35-66, wherein said formulation is suitable for use as an anti-shock therapeutic.

68. The formulation according to any of claims 36, 37, 40-42, 45-47 and 50-67, wherein said trauma comprises one or more organ system failure.

69. The formulation according to claim 68, wherein said trauma comprises liver and/or kidney failure.

70. A method of treating shock subsequent to trauma and hemorrhage in a mammalian subject, comprising administering to the subject a therapeutically effective amount of extracellular vesicles derived from cardiosphere-derived cells (CDC-EVs).

71. The method of claim 70, wherein the extracellular vesicles comprise exosomes.

72. The method of claim 70, wherein the mammal is a human.

73. The method of claim 70, wherein the trauma comprises polytrauma.

74. The method of claim 70, wherein the trauma comprises organ trauma.

75. The method of claim 73, wherein the organ comprises liver, kidney, heart, intestines, colon and/or brain.

76. The method of claim 73, wherein the trauma further comprises trauma to the skeletal system.

77. The method of claim 70, wherein the trauma results from gunshot or explosion.

78. The method of claim 70, wherein the CDC-EVs are administered within about 1 hour, about 2 hours, about 3 hours, about 4 hours, 5 hours, 6 hours, about 7 hours, about 8 hours, about 9 hours, about 10 hours, about 11 hours or about 12 hours after the trauma.

79. The method of claim 70, wherein the administration reduces risks of subject mortality compared with non-administration.

80. The method of claim 70, wherein the administration reduces serum levels of one or more of lactate, glucose and creatinine.

81. The method of claim 70, wherein the subject is a military personnel.

82. The method of claim 81, wherein the military personnel sustained the trauma during combat.
